(19) United States
(12) Patent Application Publication
(10) Pub. No.: US 2009/0214544 A1
(43) Pub. Date: Aug. 27, 2009

(54) Method of treating cd30 positive lymphomas

(75) Inventors: Steven Fischkoff, Short Hills, NJ (US); Michael Yellin, Montclair, NJ (US)

Correspondence Address: LAHIVE & COCKFIELD, LLP;FLOOR 30, SUITE 3000, ONE POST OFFICE SQUARE, BOSTON, MA, 02109, US

(73) Assignee: MEDAREX, Princeton, NJ (US)

(21) Appl. No.: 11/918,272

(22) PCT Filed: Apr. 25, 2006
(86) PCT No.: PCT/US2006/015371

§ 371 Date: Feb. 20, 2009

Related U.S. Application Data
(60) Provisional application No. 60/674,860, filed on Apr. 25, 2005.

(51) Int. Cl. A61K 39/395

(52) U.S. Cl. 424/138.1; 424/142.1; 424/143.1

(57) ABSTRACT

Methods for treating lymphomas characterized by expression of CD30 using anti-CD30 antibodies and steroids in combination are disclosed.

### BACKGROUND OF THE INVENTION

The CD30 cell surface molecule is a member of the tumor necrosis factor receptor (TNF-R) superfamily. This family of molecules has variable homology among its members and includes nerve growth factor receptor (NGFR), CD120(a), CD120(b), CD27, CD40, CD95, OX40, Fas, TNF-R1, and TNF-R2, which are key regulatory molecules that transduce signals from the environment into the cell modulating immune responses (Croft et al., Cytokine Growth Factor Rev. 14: 265-273, 2003; Cheng et al., Immunol. Res. 27: 287-294, 2003). These molecules are typically characterized by the presence of multiple cysteine-rich repeats in the extracytoplasmic region (de Bruin et al., Leukemia 9:1620-1627, 1995). Members of this family are considered crucial for regulating proliferation and differentiation of lymphocytes.

CD30 is a type I transmembrane glycoprotein with six (human) or three (murine and rat) cysteine-rich repeats with a central hinge sequence. CD30 exists as a 120 kDa membrane molecule which develops from an intercellular precursor protein of 90 kDa. It is shed from the cell surface as a soluble protein (sCD30) of approximately 90 kDa. Shedding of sCD30 occurs as an active process of viable CD30 cells and is not merely caused by the release from dying or dead cells. cDNAs encoding the CD30 protein have been cloned from expression libraries of the HLTV-1 human T-cell line HUT-102 by immunoscreening with monoclonal antibodies Ki-1 and Ber-H2 (Schwab et al., Nature 299:65, 1982). The mouse and rat CD30 cDNA has been found to encode 498 and 493 amino acids, respectively. Human CD30 cDNA encodes an additional 90 amino acids, partially duplicated from one of the cysteine rich domains. The CD30 gene has been mapped to 1p36 in humans and 5q36.2 in rats.

CD30 is preferentially expressed by activated lymphoid cells. The cell surface receptor was originally identified by the monoclonal antibody Ki-1, which is reactive with antigens expressed on Hodgkin and Reed-Sternberg cells of Hodgkin's disease (Schwab et al., 1982). Accordingly, CD30 is widely used as a clinical marker for Hodgkin's lymphoma and related hematological malignancies (Froese et al., J. Immunol. 139:2081, 1987; Carde et al., Eur. J. Cancer 26:474, 1990). It was later determined that stimulation of CD30 in lymphoid cells has been shown to induce pleiotropic biological effects, including proliferation, activation, differentiation and cell death, depending on cell type, stage of differentiation and presence of other stimuli (Gruss, et al., Blood 83:2045-2056, 1994). It is believed that the overexpression of CD30 receptor on the malignant cells contributes to survival and apoptosis resistance due to the activation of NF-kB in HD-derived cells (Izban et al., Mod. Pathol. 14: 297-310, 2001); Horie et al., Int. J. Hematol. 77: 37-47, 2003; Horie et al., Oncogene, 21: 2493-2503, 2002).

CD30 has been shown to be expressed on a subset of non-Hodgkin's lymphomas (NHL), including Burkitt's lymphoma, anaplastic large-cell lymphomas (ALCL), cutaneous T-cell lymphomas, nodular small cleaved-cell lymphomas, lymphocytic lymphomas, peripheral T-cell lymphomas, Lennert's lymphomas, immunoblastic lymphomas, T-cell leukemia/lymphomas (ATLL), adult T-cell leukemia (T-ALL), and entroblastic/centrocytic (cb/cc) follicular lymphomas (Stein et al., Blood 66:848, 1985; Miettinen, Arch. Pathol. Lab. Med. 116:1197, 1992; Piris et al., Histopathology 17:211, 1990; Burns et al., Am. J. Clin. Pathol. 93:327, 1990; and Eckert et al., Am. J. Dermatopathol. 11:345, 1989), as well as several virally-transformed lines such as human T-Cell Lymphotrophic Virus I or II transformed T-cells, and Epstein-Barr Virus transformed B-cells (Stein et al., 1985; Andreesen et al., Blood 63:1299, 1984). In addition, CD30 expression has been documented in embryonal carcinomas, nonembryonal carcinomas, malignant melanomas, mesenchymal tumors, and myeloid cell lines and macrophages at late stages of differentiation (Schwarting et al., Blood 74:1678, 1989; Pallesen et al., Am J. Pathol. 133:446, 1988; Mechtersheimer et al., Cancer 66:1732, 1990; Andreesen et al., Am. J. Pathol. 134:187, 1989).

Approximately 20 to 30% of HD patients having advanced age or HD stage will relapse after first line therapy. Of these patients, salvage therapy consisting of high dose drug therapy combined with autologous stem cell transplant can cure an additional 40-60%. Numerous single agent regimens, e.g., oral etoposide, chlorambucil, vinblastine, gemcitabine, vinorelbine, can palliate patients who fail transplant or are ineligible for transplant for months or years (Devizzi et al., Annals of Oncology 5: 817-820, 1994). More recently developed salvage therapies, such as proteasome inhibitors, anti-CD30 antibodies, and combination regimens, e.g., doxil, navelbine and gemcitabine, remain largely ineffective against treating CD30 positive lymphomas with few exceptions.

Since the percentage of CD30-positive cells in normal individuals is quite small, the expression of CD30 in tumor cells renders it an important target for antibody mediated therapy to specifically target therapeutic agents against CD30-positive neoplastic cells (Chaiarle et al., Clin. Immunol. 90(2):157-164, 1999). While the results obtained to date clearly establish CD30 as a useful target for immunotherapy, they also show that currently available murine and chimeric antibodies do not constitute ideal therapeutic agents. The fully human anti-CD30 monoclonal antibody 5F11 has shown efficacy against ALCL and various HD-derived cell lines in vitro and in vivo (Borchmann et al., Blood 102: 3737-3742, 2003). However, despite the improved efficacy of the fully human antibody over murine and chimeric anti-CD30 antibodies, variations in the sensitivity of CD30 positive target cells to 5F11 have been observed. Improvements in the ability of antibody therapies to kill CD30-expressing cells responsible for CD30 positive lymphomas is desirable.

Accordingly, there is a need for improved antibody therapies which are effective at treating and/or preventing diseases mediated by CD30.

### SUMMARY OF THE INVENTION

The present invention provides methods for treating patients having CD30 positive lymphomas (B and T cell) by administering a therapeutically effective amount of an anti-CD30 monoclonal antibody in combination with a glucocorticosteroid. Methods of the present invention provide improved efficacy of anti-CD30 antibody therapy in treating CD30 positive lymphomas. For example, the claimed methods can provide a synergistic or additive effect between a glucocorticosteroid and an anti-CD30 monoclonal antibody which results in increased efficacy of the antibody therapy, e.g., increased efficacy in inhibiting or killing tumor cells which express CD30 when compared to the administration of the antibody or the glucocorticosteroid alone.

In one embodiment, the anti-CD30 antibody is administered concurrently with the steroid, in dosages of antibody ranging from 0.0001 mg/kg to 100 mg/kg and dosages of glucocorticosteroid ranging from 0.01 mg/kg to 10,000 mg hydrocortisone equivalent per dose.

In another embodiment, the anti-CD30 antibody is administered prior to administration of the steroid, in dosages of antibody ranging from 0.0001 mg/kg to 100 mg/kg and dosages of glucocorticosteroid ranging from 0.01 mg/kg to 10,000 mg hydrocortisone equivalent per dose. In one aspect of this embodiment, the antibody is administered for a period of time prior to initiating steroid therapy, and antibody therapy is continued during steroid therapy.

In another embodiment, the anti-CD30 antibody is administered subsequent to administration of steroid, in dosages of antibody ranging from 0.0001 mg/kg to 100 mg/kg and dosages of glucocorticosteroid ranging from 0.01 mg/kg to 10,000 mg hydrocortisone equivalent per dose. In one aspect of this embodiment, the steroid is administered for a period of time prior to initiating antibody therapy, and steroid therapy is continued during antibody therapy.

Other features and advantages of the instant invention be apparent from the following detailed description and examples which should not be construed as limiting. The contents of all references, patents and published patent applications cited throughout this application are expressly incorporated herein by reference

### DETAILED DESCRIPTION OF THE INVENTION

In order that the present invention may be more readily understood, certain terms are first defined. Additional definitions are set forth throughout the detailed description.

The terms “CD30” and “CD30 antigen” are used interchangeably herein, and include any variants, isoforms and species homologs of human CD30 which are naturally expressed by cells. In a preferred embodiment, binding of an antibody of the invention to the CD30-antigen inhibits the growth of cells expressing CD30 (e.g., a tumor cell) by inhibiting or blocking binding of CD30 ligand to CD30. The term “CD30 ligand” encompasses all (e.g., physiological) ligands for CD30. In a preferred embodiment, the CD30 ligand is CD30L, CD153, TRAF1, TRAF2, TRAF3 or TRAF5. In another preferred embodiment, binding of an antibody of the invention to the CD30-antigen mediates effector cell phagocytosis and/or killing of cells expressing CD30. In yet another preferred embodiment, binding of an antibody of the invention to the CD30-antigen mediates effector cell ADCC of cells expressing CD30.

As used herein, the term “inhibits growth” (e.g., referring to cells) is intended to include any measurable decrease in the growth of a cell when contacted with an anti-CD30 antibody as compared to the growth of the same cell not in contact with an anti-CD30 antibody, e.g., the inhibition of growth of a cell by at least about 10%, 20%, 30%, 40%, 50%, 60%, 70%, 80%, 90%, 99%, or 100%.

The term “increased efficacy” is intended to include an increase in an anti-CD30 antibody's effect on tumor cells which express CD30. As described herein, such increased efficacy can result from the interaction of the antibody with a glucocorticosteroid so that their combined effect on a tumor cell (e.g., inhibition of cell growth) is greater than their individual effects alone (i.e., so that the effects are “additive”). The interaction of the antibody and glucocorticosteroid can also be “synergistic” in that their combined effect is greater than the sum of their individual effects. Measurement of these additive and synergistic effects between an anti-CD30 monoclonal antibody and a glucocorticosteroid on a tumor cell are known in the art and may be calculated, for example, by the following method. A comparison can be made between the mean objective response rate, i.e., the sum of the complete response rate (the number of patients which exhibit a complete response divided by the total number of patients) and the partial response rate (the number of patients which exhibit a partial response divided by the total number of patients), of a population of patients with a CD30 positive lymphoma treated with either the anti-CD30 antibody or glucocorticosteroid alone and a population of patients treated with the combination of the anti-CD30 antibody and the glucocorticosteroid. Alternatively, calculation of the additive and synergistic effects can be determined by comparing the patient populations described above with respect to time to progression, time to treatment failure and time of progression free survival. Methods also exist to measure the additive and synergistic effects in animal models where CD30 positive lymphoma cell lines are either injected beneath the skin to form a tumor, or are injected into the bloodstream to create a disseminated malignancy model. Furthermore, additional methods can measure the additive and synergistic effects by treating CD30 positive lymphoma cell lines growing in vitro with the anti-CD30 antibody, the glucocorticosteroid or the combination of the two.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

An “isolated antibody”, as used herein, is intended to refer to an antibody that is substantially free of other antibodies having different antigenic specificities (e.g., an isolated antibody that specifically binds CD30 is substantially free of antibodies that specifically bind antigens other than CD30). An isolated antibody that specifically binds CD30 may, however, have cross-reactivity to other antigens, such as CD30 molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g. IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody. The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to human CD30” is intended to refer to an antibody that binds to human CD30 with a K_{D} of 1×10⁻⁷ M or less, more preferably 5×10⁻⁸ M or less, more preferably 3×10⁻⁸ M or less, more preferably 1×10⁻⁸ M or less, even more preferably 1×10⁻⁹ M or less.

The term “K_{assoc}” or “Kₐ”, as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore® system.

As used herein, the term “high affinity” for an IgG antibody refers to an antibody having a K_{D} of 10⁻⁸ M or less, more preferably 10⁻⁹ M or less and even more preferably 10⁻¹⁰ M or less for a target antigen. However, “high affinity” binding can vary for other antibody isotypes. For example, “high affinity” binding for an IgM isotype refers to an antibody having a K_{D} of 10⁻⁷ M or less, more preferably 10⁻⁸ M or less, even more preferably 10⁻⁹ M or less.

As used herein, the terms “subject” and “patient” are used interchangeably and can refer to any human or nonhuman animal. The term “nonhuman animal” includes all vertebrates, e.g., mammals and non-mammals, such as nonhuman primates, sheep, dogs, cats, horses, cows, chickens, amphibians, reptiles, etc. In a particular embodiment of the present invention the patient is a human.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Bach heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g., CD30). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., Science 242:423-426, 1988; and Huston et al., Proc. Natl. Acad. Sci. USA 85:5879-5883, 1988). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

As used herein, the terms “inhibits binding” and “blocks binding” (e.g., referring to inhibition/blocking of binding of CD30 ligand to CD30. Inhibition/blocking are used interchangeably and encompass both partial and complete inhibition/blocking. The inhibition/blocking of CD30 preferably reduces or alters the normal level or type of activity that occurs when CD30 binding occurs without inhibition or blocking, e.g., inhibition of CD30 induced proliferation. Inhibition and blocking are also intended to include any measurable decrease in the binding affinity of CD30 when in contact with an anti-CD30 antibody as compared to CD30 not in contact with an anti-CD30 antibody, e.g., the blocking of CD30 to its receptor by at least about 10%, 20%, 30%, 40%, 50%, 60%, 70%, 80%, 90%, 99% or 100%.

The term “bispecific molecule” is intended to include any agent, e.g., a protein, peptide, or protein or peptide complex, which has two different binding specificities. For example, the molecule may bind to, or interact with, (a) a cell surface antigen and (b) an Fc receptor on the surface of an effector cell. The term “multispecific molecule” or “heterospecific molecule” is intended to include any agent, e.g., a protein, peptide, or protein or peptide complex, which has more than two different binding specificities. For example, the molecule may bind to, or interact with, (a) a cell surface antigen, (b) an Fc receptor on the surface of an effector cell, and (c) at least one other component. Accordingly, the invention includes, but is not limited to, bispecific, trispecific, tetraspecific, and other multispecific molecules which are directed to cell surface antigens, such as CD30, and to other targets, such as Fc receptors on effector cells.

The term “bispecific antibodies” also includes diabodies. Diabodies are bivalent, bispecific antibodies in which the V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g. Holliger, P., et al. (1993) Proc. Natl. Acad. Sci. USA 90:6444-6448; Poljak, R. J., et al. (1994) Structure 2:1121-1123). As used herein, the term “heteroantibodies” refers to two or more antibodies, antibody-binding fragments (e.g., Fab), derivatives therefrom, or antigen binding regions linked together, at least two of which have different specificities. These different specificities include a binding specificity for an Fc receptor on an effector cell, and a binding specificity for an antigen or epitope on a target cell, e.g., a tumor cell.

As used herein, a “heterologous antibody” is defined in relation to the transgenic non-human organism producing such an antibody. This term refers to an antibody having an amino acid sequence or an encoding nucleic acid sequence corresponding to that found in an organism not consisting of the transgenic non-human animal, and generally from a species other than that of the transgenic non-human animal.

As used herein, a “heterohybrid antibody” refers to an antibody having a light and heavy chains of different organismal origins. For example, an antibody having a human heavy chain associated with a murine light chain is a heterohybrid antibody. Examples of heterohybrid antibodies include chimeric and humanized antibodies, discussed supra.

As used herein, “glycosylation pattern” is defined as the pattern of carbohydrate units that are covalently attached to a protein, more specifically to an immunoglobulin protein. A glycosylation pattern of a heterologous antibody can be characterized as being substantially similar to glycosylation patterns which occur naturally on antibodies produced by the species of the nonhuman transgenic animal, when one of ordinary skill in the art would recognize the glycosylation pattern of the heterologous antibody as being more similar to said pattern of glycosylation in the species of the nonhuman transgenic animal than to the species from which the CH genes of the transgene were derived.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “rearranged” as used herein refers to a configuration of a heavy chain or light chain immunoglobulin locus wherein a V segment is positioned immediately adjacent to a D-J or J segment in a conformation encoding essentially a complete V_{H} or V_{L} domain, respectively. A rearranged immunoglobulin gene locus can be identified by comparison to germline DNA; a rearranged locus will have at least one recombined heptamer/nonamer homology element.

The term “unrearranged” or “germline configuration” as used herein in reference to a V segment refers to the configuration wherein the V segment is not recombined so as to be immediately adjacent to a D or J segment.

### Anti-CD30 Antibodies

Antibodies against CD30 are well-known in the art, e.g., 5F11, 17G1 and 2H9 (US Patent Application Publication No. 2004/0006215); HeFi-1 and AC-10 (International Patent Publication Nos. WO 2002/43661, WO 2004/010957, and WO 2005/001038; Hecht et al., 1985, J. Immunol. 134:4231-4236; Tian et al. (1995) Cancer Res. 55:5335; Koon et al., 2000, Current Opinion in Oncology 12:588; Wahl et al., 2002, Cancer Res. 62: 3736-3742); C10 (Bowen et al., 1993, J. Immunol. 151:5896-5906); M44 (Smith et al., 1993, Cell 73:1349); Ber-H2 (Schwarting et al., 1989, Blood 74:1678-89 17G1 and 2H9), HRS-1, HRS-3 and HRS-4 (Pfreundschuh et al., 1988, Anticancer Res. 8:217-244; Pohl et al., 1993, Int. J. Cancer 54: 418-25), Ki-1, Ki-2, Ki-3, Ki-4, Ki-5, Ki-6 and Ki-7 (Schwaab et al., 1982, 299:65-67; Horn-Lohrens et al., 1995, Int. J. cancer 60: 539-544; U.S. Pat. No. 6,033,876); IRac (Hsu et al., 1987, J. Natl. Cancer inst. 79:1091-1097; Engert et al., 1990, Cancer Res. 50:2929-2935), M67 (Smith et al., 1993); and T6, T13, T14, T24 and T25 (Nagata, 2002, Clin. Cancer Res. 8:2345-2355). Each of the cited publications are incorporated by reference in their entireties.

Preferably, an antibody used in a method of the present invention is chimeric, humanized or human. In a particular embodiment, the antibody is a fully human antibody. Preferred antibodies for use in a method of the invention are characterized by particular functional features or properties of the antibodies. For example, the antibodies bind specifically to human CD30 with high affinity, and preferably exhibit one or more of the following characteristics:

a) a binding affinity to CD30 with an affinity constant of at least about 10⁷ M⁻¹, preferably about 10⁸ M⁻¹, and more preferably, about 10⁹ M⁻¹ to 10¹⁰ M⁻¹ or higher;

b) an association constant (K_{assoc}) with CD30 of at least about 10³, more preferably about 10⁴ and most preferably about 10⁵ M⁻¹S⁻¹;

c) a dissociation constant (K_{dis}) from CD30 of about 10⁻³ s⁻¹, preferably about 10⁻⁴ s⁻¹, more preferably, 10⁻⁵ s⁻¹, and most preferably, 10⁻⁶ s⁻¹;

d) the ability to opsonize a cell expressing CD30;

e) the ability to inhibit growth and/or mediate phagocytosis and killing of cells expressing CD30 (e.g., a tumor cell) in the presence of human effector cells at a concentration of about 10 μg/ml or less (e.g., in vitro); or

f) the ability to bind to CD30 and inhibit CD30 function (e.g. CD30 mediated effects) by partially or completely blocking a CD30 ligand binding to CD30 (examples of CD30 ligands include CD153, TRAF1, TRAF2, TRAF3 and TRAF5).

Preferably, the antibody binds to human CD30 with a K_{D} of 5×10⁻⁹ M or less, binds to human CD30 with a K_{D} of 4×10⁻⁹ M or less, binds to human CD30 with a K_{D} of 3.5×10⁻⁹ M or less, binds to human CD30 with a K_{D} of 3×10⁻⁹ M or less or binds to human CD30 with a K_{D} of 2.8×10⁻⁹ M or less.

Standard assays to evaluate the binding ability of the antibodies toward CD30 are known in the art, including for example, ELISA, Western blot and RIA. Suitable assays are described in detail in the Examples. The binding kinetics (e.g., binding affinity) of the antibodies also can be assessed by standard assays known in the art, such as by Biacore analysis.

### Monoclonal Antibodies 17G1, 2H9 and 5F11

Examples of certain preferred antibodies for use in the invention include the human monoclonal antibodies 17G1, 2H9 and 5F11, which are characterized and described in US Patent Application Publication No. 2004/0006215, which is hereby incorporated by reference in its entirety. Additional preferred antibodies include The V_{H} amino acid sequences of 17G1, 2H9 and 5F11 are shown in SEQ ID NOs: 2, 6 and 10, respectively. The V_{L} amino acid sequences of 17G1, 2H9 and 5F11 are shown in SEQ ID NOs: 4, 8 and 12, respectively.

Given that each of these antibodies can bind to CD30, the V_{H} and V_{L} sequences can be “mixed and matched” to create other anti-CD30 binding molecules for use in a method of the invention. CD30 binding of such “mixed and matched” antibodies can be tested using the binding assays described above and in the Examples (e.g., ELISAs). Preferably, when V_{H} and V_{L} chains are mixed and matched, a V_{H} sequence from a particular V_{H}/V_{L} pairing is replaced with a structurally similar V_{H} sequence. Likewise, preferably a V_{L} sequence from a particular V_{H}/V_{L} pairing is replaced with a structurally similar V_{L} sequence.

Accordingly, in one aspect, a method of the invention can employ a monoclonal antibody, or antigen binding portion thereof comprising:

(a) a heavy chain variable region comprising an amino acid sequence selected from the group consisting of SEQ ID NOs: 2, 6 and 10; and

(b) a light chain variable region comprising an amino acid sequence selected from the group consisting of SEQ ID NOs: 4, 8 and 12;

wherein the antibody specifically binds CD30, preferably human CD30. Preferred heavy and light chain combinations include:

(a) a heavy chain variable region comprising the amino acid sequence of SEQ ID NO: 2; and (b) a light chain variable region comprising the amino acid sequence of SEQ ID NO: 4; or

(b) a heavy chain variable region comprising the amino acid sequence of SEQ ID NO: 6; and (b) a light chain variable region comprising the amino acid sequence of SEQ ID NO: 8; or

(c) a heavy chain variable region comprising the amino acid sequence of SEQ ID NO: 10; and (b) a light chain variable region comprising the amino acid sequence of SEQ ID NO: 12.

In another aspect, antibodies that comprise the heavy chain and light chain CDR1s, CDR2s and CDR3s of 17G1, 2H9 and 5F11, or combinations thereof can be used in the present method. The amino acid sequences of the V_{H} CDR1s of 17G1, 2H9 and 5F11 are shown in SEQ ID NOs: 16, 28 and 40. The amino acid sequences of the V_{H} CDR2s of 17G1, 2H9 and 5F11 are shown in SEQ ID NOs: 17, 29 and 41. The amino acid sequences of the V_{H} CDR3s of 17G1, 2H9 and 5F11 are shown in SEQ ID NOs: 18, 30 and 42. The amino acid sequences of the Vₖ CDR1s of 17G1, 2H9 and 5F11 are shown in SEQ ID NOs: 22, 34 and 46. The amino acid sequences of the Vₖ CDR2s of 17G1, 2H9 and 5F11 are shown in SEQ ID NOs: 23, 35 and 47. The amino acid sequences of the Vₖ CDR3s of 17G1, 2H9 and 5F11 are shown in SEQ ID NOs: 24, 36 and 48. The CDR regions are delineated using the Kabat system (Kabat, E. A., et al. (1991) Sequences of Proteins of Immunological Interest, Fifth Edition, U.S. Department of Health and Human Services, NIH Publication No. 91-3242).

Given that each of these antibodies can bind to CD30 and that antigen-binding specificity is provided primarily by the CDR1, CDR2, and CDR3 regions, the V_{H} CDR1, CDR2, and CDR3 sequences and Vₖ CDR1, CDR2, and CDR3 sequences can be “mixed and matched” (i.e., CDRs from different antibodies can be mixed and match, although each antibody must contain a V_{H} CDR1, CDR2, and CDR3 and a Vₖ CDR1, CDR2, and CDR3) to create other anti-CD30 binding molecules for use in the invention. CD30 binding of such “mixed and matched” antibodies can be tested using the binding assays described above and in the Examples (e.g., ELISAs, Biacore analysis). Preferably, when V_{H} CDR sequences are mixed and matched, the CDR1, CDR2 and/or CDR3 sequence from a particular V_{H} sequence is replaced with a structurally similar CDR sequence(s). Likewise, when Vₖ CDR sequences are mixed and matched, the CDR1, CDR2 and/or CDR3 sequence from a particular Vₖ sequence preferably is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences disclosed herein for monoclonal antibodies 17G1, 2H9 and 5F11.

Accordingly, in another aspect, a method of the invention can employ an isolated monoclonal antibody, or antigen binding portion thereof comprising:

(a) a heavy chain variable region CDR1 comprising an amino acid sequence selected from the group consisting of SEQ ID NOs: 16, 28 and 40;

(b) a heavy chain variable region CDR2 comprising an amino acid sequence selected from the group consisting of SEQ ID NOs: 17, 29 and 41;

(c) a heavy chain variable region CDR3 comprising an amino acid sequence selected from the group consisting of SEQ ID NOs: 18, 30 and 42;

(d) a light chain variable region CDR1 comprising an amino acid sequence selected from the group consisting of SEQ ID NOs: 22, 34 and 46;

(e) a light chain variable region CDR2 comprising an amino acid sequence selected from the group consisting of SEQ ID NOs: 23, 35 and 47; and

(f) a light chain variable region CDR3 comprising an amino acid sequence selected from the group consisting of SEQ ID NOs: 24, 36 and 48;

wherein the antibody specifically binds CD30, preferably human CD30.

In a preferred embodiment, the antibody comprises:

(a) a heavy chain variable region CDR1 comprising SEQ ID NO: 16;

(b) a heavy chain variable region CDR2 comprising SEQ ID NO: 17;

(c) a heavy chain variable region CDR3 comprising SEQ ID NO: 18;

(d) a light chain variable region CDR1 comprising SEQ ID NO: 22;

(e) a light chain variable region CDR2 comprising SEQ ID NO: 23; and

(f) a light chain variable region CDR3 comprising SEQ ID NO: 24.

In another preferred embodiment, the antibody comprises:

(a) a heavy chain variable region CDR1 comprising SEQ ID NO: 28;

(b) a heavy chain variable region CDR2 comprising SEQ ID NO: 29;

(c) a heavy chain variable region CDR3 comprising SEQ ID NO: 30;

(d) a light chain variable region CDR1 comprising SEQ ID NO: 34;

(e) a light chain variable region CDR2 comprising SEQ ID NO: 35; and

(f) a light chain variable region CDR3 comprising SEQ ID NO: 36.

In yet another preferred embodiment, the antibody comprises:

(a) a heavy chain variable region CDR1 comprising SEQ ID NO: 40;

(b) a heavy chain variable region CDR2 comprising SEQ ID NO: 41;

(c) a heavy chain variable region CDR3 comprising SEQ ID NO: 42;

(d) a light chain variable region CDR1 comprising SEQ ID NO: 46;

(e) a light chain variable region CDR2 comprising SEQ ID NO: 47; and

(f) a light chain variable region CDR3 comprising SEQ ID NO: 48.

### Use of Antibodies Having Particular Germline Sequences

In certain embodiments, an antibody used in a method of the invention comprises a heavy chain variable region from a particular germline heavy chain immunoglobulin gene and/or a light chain variable region from a particular germline light chain immunoglobulin gene.

For example, in preferred embodiments, the monoclonal antibody, or an antigen-binding portion thereof, comprises a heavy chain variable region that is the product of or derived from a human V_{H} 4-34 gene or a human V_{H} 3-11 gene, wherein the antibody specifically binds CD30. In other preferred embodiments, the monoclonal antibody, or an antigen-binding portion thereof, comprises a light chain variable region that is the product of or derived from a human V_{K} L15 gene, a human V_{K} A27 gene or a human V_{K} L6 gene, wherein the antibody specifically binds CD30. In yet another preferred embodiment, the invention provides an isolated monoclonal antibody, or antigen-binding portion thereof, wherein the antibody:

(a) comprises a heavy chain variable region that is the product of or derived from a human V_{H} 4-34 or 3-11 gene (which genes encode the amino acid sequences set forth in SEQ ID NO: 49 and 51, respectively);

(b) comprises a light chain variable region that is the product of or derived from a human V_{K} L15 or V_{K} A27 or V_{K} L6 gene (which genes encode the amino acid sequences set forth in SEQ ID NO: 50, 52 and 53, respectively); and

(c) specifically binds to CD30, preferably human CD30.

An example of an antibody having V_{H} and V_{K} of V_{H} 4-34 and V_{K} L15, respectively, is 5F11. An example of an antibody having V_{H} and V_{K} of V_{H} 3-11 and V_{K} A27, respectively, is 17G1. An example of an antibody having a V_{H} and V_{K} of V_{H} 4-34 and V_{K} L6, respectively, is 2H9.

As used herein, a human antibody comprises heavy or light chain variable regions that is “the product of” or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody. A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

### Homologous Antibodies

In yet another embodiment, an antibody useful in the invention comprises heavy and light chain variable regions comprising amino acid sequences that are homologous to the amino acid sequences of the preferred antibodies described herein, and wherein the antibodies retain the desired functional properties of the preferred anti-CD30 antibodies.

For example, monoclonal antibodies, or antigen binding portion thereof, useful in a method of the invention comprise a heavy chain variable region and a light chain variable region, wherein:

(a) the heavy chain variable region comprises an amino acid sequence that is at least 80% homologous to an amino acid sequence selected from the group consisting of SEQ ID NOs: 2, 6 and 10;

(b) the light chain variable region comprises an amino acid sequence that is at least 80% homologous to an amino acid sequence selected from the group consisting of SEQ ID NOs: 4, 8 and 12;

(c) the antibody binds to human CD30 with a K_{D} of 1×10⁻⁸ M or less;

(d) the antibody has an association constant (K_{assoc}) with CD30 of at least about 10³, more preferably about 10⁴ and most preferably about 10⁵ M⁻¹S⁻¹;

(e) the antibody has a dissociation constant (K_{dis}) from CD30 of about 10⁻³ s⁻¹ preferably about 10⁴ s⁻¹, more preferably, 10⁻⁵ s⁻¹, and most preferably, 10⁻⁶ s⁻¹;

(f) the antibody has the ability to opsonize a cell expressing CD30;

(g) the antibody has the ability to inhibit growth and/or mediate phagocytosis and killing of cells expressing CD30 (e.g., a tumor cell) in the presence of human effector cells at a concentration of about 10 μg/ml or less (e.g., in vitro); or

(h) the antibody has the ability to bind to CD30 and inhibit CD30 function (e.g., CD30 mediated effects) by partially or completely blocking CD30 ligand binding to CD30 (examples of CD30 ligands include CD153, TRAF1, TRAF2, TRAF3 and TRAF5).

In various embodiments, the antibody can be, for example, a human antibody, a humanized antibody or a chimeric antibody.

In other embodiments, the V_{H} and/or V_{L} amino acid sequences may be 85%, 90%, 95%, 96%, 97%, 98% or 99% homologous to the sequences set forth above. An antibody having V_{H} and V_{L} regions having high (i.e., 80% or greater) homology to the V_{H} and V_{L} regions of the sequences set forth above, can be obtained by mutagenesis (e.g., site-directed or PCR-mediated mutagenesis) of nucleic acid molecules encoding SEQ ID NOs: 1, 3, 5, 7, 9 and 11, followed by testing of the encoded altered antibody for retained function (i.e., the functions set forth in (c) and (d) above) using the functional assays described herein.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

Antibodies with Conservative Modifications

In certain embodiments, an antibody of the invention comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein (e.g., 17G1, 2H9 or 5F11), or conservative modifications thereof, and wherein the antibodies retain the desired functional properties of the anti-CD30 antibodies of the invention. Accordingly, the invention provides an isolated monoclonal antibody, or antigen binding portion thereof, comprising a heavy chain variable region comprising CDR1, CDR2, and CDR3 sequences and a light chain variable region comprising CDR1, CDR2, and CDR3 sequences, wherein:

(a) the heavy chain variable region CDR3 sequence comprises an amino acid sequence selected from the group consisting of amino acid sequences of SEQ ID NOs: 18, 30 and 42, and conservative modifications thereof;

(b) the light chain variable region CDR3 sequence comprises an amino acid sequence selected from the group consisting of amino acid sequence of SEQ ID NOs: 24, 36 and 48, and conservative modifications thereof;

(c) the antibody binds to human CD30 with a K_{D} of 1×10⁻⁸ M or less;

(d) the antibody has an association constant (K_{assoc}) with CD30 of at least about 10³, more preferably about 10⁴ and most preferably about 10⁵ M⁻¹S⁻¹;

(e) the antibody has a dissociation constant (K_{dis}) from CD30 of about 10⁻³ s⁻¹, preferably about 10⁻⁴ s⁻¹, more preferably, 10⁻⁵ s⁻¹, and most preferably, 10⁻⁶ s⁻¹;

(f) the antibody has the ability to opsonize a cell expressing CD30;

(g) the antibody has the ability to inhibit growth and/or mediate phagocytosis and killing of cells expressing CD30 (e.g., a tumor cell) in the presence of human effector cells at a concentration of about 10 μg/ml or less (e.g., in vitro); or

(h) the antibody has the ability to bind to CD30 and inhibit CD30 function (e.g., CD30 mediated effects) by partially or completely blocking CD30 ligand binding to CD30 (examples of CD30 ligands include CD153, TRAF1, TRAF2, TRAF3 and TRAF5).

In a preferred embodiment, the heavy chain variable region CDR2 sequence comprises an amino acid sequence selected from the group consisting of amino acid sequences of SEQ ID NOs: 17, 29 and 41, and conservative modifications thereof; and the light chain variable region CDR2 sequence comprises an amino acid sequence selected from the group consisting of amino acid sequences of SEQ ID NOs: 23, 35 and 47, and conservative modifications thereof. In another preferred embodiment, the heavy chain variable region CDR1 sequence comprises an amino acid sequence selected from the group consisting of amino acid sequences of SEQ ID NOs: 16, 28 and 40, and conservative modifications thereof; and the light chain variable region CDR1 sequence comprises an amino acid sequence selected from the group consisting of amino acid sequences of SEQ ID NOs: 22, 34 and 46, and conservative modifications thereof.

In various embodiments, the antibody can be, for example, human antibodies, humanized antibodies or chimeric antibodies.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g. tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the functions set forth in (c) through (1) above) using the functional assays described herein.

Antibodies that Bind to the Same Epitope as Anti-CD30 Antibodies of the Invention

In addition to the antibodies described herein, it is contemplated that a method of the invention can employ antibodies that bind to the same cluster (A, B or C), or more preferably to the same epitope, on human CD30 as any of the CD30 monoclonal antibodies described (i.e., antibodies that have the ability to cross-compete for binding to CD30 with any of the monoclonal antibodies described herein, e.g., 17G1, 2H9, 5F11, M44, HeFi-1, C10, AC10, Ber-H2, HRS-1, HRS-3, HRS-4, Ki-1, Ki-2, Ki-3, Ki-4, Ki-5, Ki-6, Ki-7, IRac, M67, T6, T13, T14, T24 and T25). Such cross-competing antibodies can be identified based on their ability to cross-compete with, e.g., 5F11 in standard CD30 binding assays. For example, BIAcore analysis, ELISA assays or flow cytometry can be used to demonstrate cross-competition with the antibodies of the current invention. The ability of a test antibody to inhibit the binding of 5F11 to human CD30 demonstrates that the test antibody can compete with such antibody for binding to human CD30 and thus binds to the same epitope on human CD30 as such antibody. In a preferred embodiment, the antibody that binds to the same epitope on human CD30 as an antibody described herein is a human monoclonal antibody, which can be prepared and isolated using methodologies well known in the art.

### Engineered and Modified Antibodies

An antibody used in the invention can be prepared using one or more of the V_{H} and/or V_{L} sequences from an antibody disclosed herein as starting material to engineer a modified antibody, which modified antibody may have altered properties from an antibody disclosed herein. An antibody can be engineered by modifying one or more residues within one or both variable regions (i.e., V_{H} and/or V_{L}), for example within one or more CDR regions and/or within one or more framework regions. Additionally, or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechinann, L. et al. (1998) Nature 332:323-327; Jones, P. et al. (1986) Nature 321:522-525; Queen, C. et al. (1989) Proc. Natl. Acad. See. USA. 86:10029-10033; U.S. Pat. No. 5,225,539 to Winter, and U.S. Pat. Nos. 5,530,101; 5,585,089; 5,693,762 and 6,180,370 to Queen et al.)

Accordingly, another embodiment of the invention pertains to an isolated monoclonal antibody, or antigen binding portion thereof, comprising a heavy chain variable region comprising CDR1, CDR2, and CDR3 sequences from 17G1, 2H9 or 5F11, but contain modifications to framework sequences. Thus, such antibodies contain the V_{H} and V_{L} CDR sequences of monoclonal antibodies 17G1, 2H9 or 5F11 yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L et al. (1994) “A Directory of Human Germ-line V_{H} Segments Reveals a Strong Bias in their Usage” Eur. J. Immunol. 24:827-836; the contents of each of which are expressly incorporated herein by reference.

Preferred framework sequences for use in the antibodies of the invention are those that are structurally similar to the framework sequences used by selected antibodies of the invention, e.g., similar to the V_{H} 4-34 framework sequences (SEQ ID NO: 49) and/or the V_{H} 3-11 framework sequences (SEQ ID NO: 51) and/or the V_{K} L15 framework sequences (SEQ ID NO: 50) and/or the Vₖ A27 framework sequence (SEQ ID NO: 52) and/or the V_{K} L6 framework sequence (SEQ ID NO: 53) used by preferred monoclonal antibodies employed in the invention. The V_{H} CDR1, CDR2, and CDR3 sequences, and the V_{K} CDR1, CDR2, and CDR3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Another type of variable region modification is to mutate amino acid residues within the V_{H} and/or V_{K} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Preferably conservative modifications (as discussed above) are introduced. The mutations may be amino acid substitutions, additions or deletions, but are preferably substitutions. Moreover, typically no more than one, two, three, four or five residues within a CDR region are altered.

Accordingly, in another embodiment, the invention provides isolated anti-CD30 monoclonal antibodies, or antigen binding portions thereof, comprising a heavy chain variable region comprising: (a) a V_{H} CDR1 region comprising an amino acid sequence selected from the group consisting of SEQ ID NOs: 16, 28 and 40, or an amino acid sequence having one, two, three, four or five amino acid substitutions, deletions or additions as compared to SEQ ID NOs: 16, 28 and 40; (b) a V_{H} CDR2 region comprising an amino acid sequence selected from the group consisting of SEQ ID NOs: 17, 29 and 41, or an amino acid sequence having one, two, three, four or five amino acid substitutions, deletions or additions as compared to SEQ ID NOs: 17, 29 and 41; (c) a V_{H} CDR3 region comprising an amino acid sequence selected from the group consisting of SEQ ID NOs: 18, 30 and 42, or an amino acid sequence having one, two, three, four or five amino acid substitutions, deletions or additions as compared to SEQ ID NOs: 18, 30 and 42; (d) a V_{K} CDR1 region comprising an amino acid sequence selected from the group consisting of SEQ ID NOs: 22, 34 and 46, or an amino acid sequence having one, two, three, four or five amino acid substitutions, deletions or additions as compared to SEQ ID NOs: 22, 34 and 46; (e) a V_{K} CDR2 region comprising an amino acid sequence selected from the group consisting of SEQ ID NOs: 23, 35 and 47, or an amino acid sequence having one, two, three, four or five amino acid substitutions, deletions or additions as compared to SEQ ID NOs: 23, 35 and 47; and (f) a V_{K} CDR3 region comprising an amino acid sequence selected from the group consisting of SEQ ID NOs: 24, 36 and 48, or an amino acid sequence having one, two, three, four or five amino acid substitutions, deletions or additions as compared to SEQ ID NOs: 24, 36 and 48.

Engineered antibodies of the invention include those in which modifications have been made to framework residues within V_{H} and/or V_{K}, e.g. to improve the properties of the antibody. Typically such framework modifications are made to decrease the immunogenicity of the antibody. For example, one known approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

In addition or alternative to modifications made within the framework or CDR regions, antibodies used in the invention can be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half life. Various approaches are possible. For example, one or more of the following mutations can be introduced: T252L, T254S, T256F, as described in U.S. Pat. No. 6,277,375 to Ward. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered Clq binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in International Patent Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgGlfor FcγR1, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1,176,195 by Hanai et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)—N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180).

Another modification of antibodies that can be used in a method of the invention that can be made includes pegylation. An antibody can be pegylated to, for example, increase the biological (e.g., serum) half life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al.

### Methods of Engineering Antibodies

As discussed above, the anti-CD30 antibodies having V_{H} and V_{K} sequences disclosed herein can be used to create new anti-CD30 antibodies by modifying the V_{H} and/or V_{K} sequences, or the constant region(s) attached thereto. Thus, in another aspect of the invention, the structural features of an anti-CD30 antibody of the invention, e.g. 17G1, 2H9 or 5F11, are used to create structurally related anti-CD30 antibodies that retain at least one functional property of the antibodies of the invention, such as binding to human CD30. For example, one or more CDR regions of 17G1, 2H9 or 5F11, or mutations thereof, can be combined recombinantly with known framework regions and/or other CDRs to create additional, recombinantly-engineered, anti-CD30 antibodies of the invention, as discussed above. Other types of modifications include those described in the previous section. The starting material for the engineering method is one or more of the V_{H} and/or V_{K} sequences provided herein, or one or more CDR regions thereof. To create the engineered antibody, it is not necessary to actually prepare (i.e., express as a protein) an antibody having one or more of the V_{H} and/or V_{K} sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, the invention provides a method for preparing an anti-CD30 antibody comprising:

(a) providing: (i) a heavy chain variable region antibody sequence comprising a CDR1 sequence selected from the group consisting of SEQ ID NOs: 16, 28 and 40, a CDR2 sequence selected from the group consisting of SEQ ID NOs: 17, 29 and 41, and/or a CDR3 sequence selected from the group consisting of SEQ ID NOs: 18, 30 and 42; and/or (ii) a light chain variable region antibody sequence comprising a CDR1 sequence selected from the group consisting of SEQ ID NOs: 22, 34 and 46, a CDR2 sequence selected from the group consisting of SEQ ID NOs: 23, 35 and 47, and/or a CDR3 sequence selected from the group consisting of SEQ ID NOs: 24, 36 and 48;

(b) altering at least one amino acid residue within the heavy chain variable region antibody sequence and/or the light chain variable region antibody sequence to create at least one altered antibody sequence; and

(c) expressing the altered antibody sequence as a protein.

Standard molecular biology techniques can be used to prepare and express the altered antibody sequence.

Preferably, the antibody encoded by the altered antibody sequence(s) is one that retains one, some or all of the functional properties of the anti-CD30 antibodies described herein, which functional properties include, but are not limited to:

(a) binds to human CD 30 with a K_{D} of 1×10⁻⁸ M or less;

(b) the light chain variable region comprises an amino acid sequence that is at least 80% homologous to an amino acid sequence selected from the group consisting of SEQ ID NOs: 4, 8 and 12;

(c) the antibody binds to human CD30 with a K_{D} of 1×10⁻⁸ M or less;

(d) the antibody has an association constant (K_{assoc}) with CD30 of at least about 10³, more preferably about 10⁴ and most preferably about 10⁵ M⁻¹ s⁻¹;

(e) the antibody has a dissociation constant (K_{dis}) from CD30 of about 10⁻³ s⁻¹, preferably about 10⁻⁴ s⁻¹, more preferably, 10⁻⁵ s⁻¹, and most preferably, 10⁻⁶ s⁻¹;

(f) the antibody has the ability to opsonize a cell expressing CD30;

(g) the antibody has the ability to inhibit growth and/or mediate phagocytosis and killing of cells expressing CO₃₀ (e.g., a tumor cell) in the presence of human effector cells at a concentration of about 10 μg/ml or less (e.g., in vitro); or

(h) the ability to bind to CD30 and inhibit CD30 function (e.g., CD30 mediated effects) by partially or completely blocking CD30 ligand binding to CD30 (examples of CD30 ligands include CD153, TRAF1, TRAF2, TRAF3 and TRAF5).

The functional properties of the altered antibodies can be assessed using standard assays available in the art and/or described herein, such as those set forth in the Examples (e.g., flow cytometry, binding assays).

In certain embodiments of the methods of engineering antibodies of the invention, mutations can be introduced randomly or selectively along all or part of an anti-CD30 antibody coding sequence and the resulting modified anti-CD30 antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

### Nucleic Acid Molecules Encoding Antibodies of the Invention

Nucleic acid molecules that encode certain of the antibodies useful in the invention are described herein (SEQ ID NOs: 1, 3, 5, 7, 9, and 11). The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid of the invention can be, for example, DNA or RNA and may or may not contain intronic sequences. In a preferred embodiment, the nucleic acid is a cDNA molecule.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Preferred nucleic acids molecules of the invention are those encoding the VH and VL sequences of the 17G1, 2H9 or 5F11 monoclonal antibodies. DNA sequences encoding the VH sequences of 17G1, 2H9 and 5F11 are shown in SEQ ID NOs: 1, 5, and 9, respectively. DNA sequences encoding the VL sequences of 17G1, 2H9 and 5F11 are shown in SEQ ID NOs: 3, 7 and 11, respectively.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgGlor IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

### Production of Monoclonal Antibodies of the Invention

Monoclonal antibodies (mAbs) useful in the present invention can be produced by a variety of techniques, including conventional monoclonal antibody methodology, e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

The preferred animal system for preparing hybridomas is the murine system. Hybridoma production in the mouse is a very well-established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In a preferred embodiment, the antibodies of the invention are human monoclonal antibodies. Such human monoclonal antibodies directed against CD30 can be generated using transgenic or transchromosomic mice carrying parts of the human immune system rather than the mouse system. These transgenic and transchromosomic mice include mice referred to herein as HuMAb-Mouse® and KM-Mouse®, respectively, and are collectively referred to herein as “human Ig mice.” These mice are well-known in the art (see e.g., Lonberg, et al. (1994) Nature 368(6474): 856-859; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). The preparation and use of HuMab mice, and the genomic modifications carried by such mice, is further described in Taylor, L. et al. (1992) Nucleic Acids Research 20:6287-6295; Chen, J. et al. (1993) International Immunology 5: 647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6: 579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.)

In another embodiment, human antibodies of the invention can be raised using a mouse that carries human immunoglobulin sequences on transgenes and transchromosomes, such as a mouse that carries a human heavy chain trarisgene and a human light chain transchromosome. Such mice, referred to herein as “KM-Mouse®”, are described in detail in PCT Publication WO 02/43478 to Ishida et al.

Still further, alternative transgenic animal systems expressing human immunoglobulin genes are available in the art and can be used to raise anti-CD30 antibodies used in the invention. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-CD30 antibodies used in the invention. For example, mice carrying both a human heavy chain transchromosome and a human light chain transchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (Kuroiwa et al. (2002) Nature Biotechnology 20:889-894) and can be used to raise anti-CD30 antibodies used in the invention.

Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies used in the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### Immunization of Human Ig Mice

Immunization of human Ig mice for raising human antibodies is described in detail in US Patent Application Publication No. 2004/0006215, which is hereby incorporated by reference in its entirety. Detailed procedures to generate fully human monoclonal antibodies to CD30 are also described therein.

### Generation of Hybridomas Producing Human Monoclonal Antibodies of the Invention

To generate hybridomas producing human monoclonal antibodies of the invention, splenocytes and/or lymph node cells from immunized mice can be isolated and fused to an appropriate immortalized cell line, such as a mouse myeloma cell line. The resulting hybridomas can be screened for the production of antigen-specific antibodies. Such methodologies are well known in the art and are described in US 2004/0006125

### Generation of Transfectomas Producing Monoclonal Antibodies of the Invention

Antibodies used in the invention can be produced in a host cell transfectoma system using, for example, a combination of recombinant DNA techniques and gene transfection methods that are well known in the art (e.g., Morrison, S. (1985) Science 229:1202), and described in detail in US 2004/0006125.

### Characterization of Binding of Human Monoclonal Antibodies to CD30

To characterize binding of human monoclonal CD30 antibodies of the invention, sera from immunized mice can be tested, for example, by ELISA. In a typical (but non-limiting) example of an ELISA protocol, microtiter plates are coated with purified CD30 at 0.25 μg/ml in PBS, and then blocked with 5% bovine serum albumin in PBS. Dilutions of plasma from CD30-immunized mice are added to each well and incubated for 1-2 hours at 37° C. The plates are washed with PBS/Tween and then incubated with a goat-anti-human IgG Fc-specific polyclonal reagent conjugated to alkaline phosphatase for 1 hour at 37° C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with CD30 immunogen. Hybridomas that bind with high avidity to CD30 will be subcloned and further characterized. One clone from each hybridoma, which retains the reactivity of the parent cells (by ELISA), can be chosen for making a 5-10 vial cell bank stored at −140° C., and for antibody purification.

To purify human anti-CD30 antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD₂₈₀ using 1.43 extinction coefficient. The monoclonal antibodies can be aliquoted and stored at −80° C.

To determine if the selected human anti-CD30 monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using CD30 coated-ELISA plates as described above. Biotinylated MAb binding can be detected with a strep-avidin-alkaline phosphatase probe.

To determine the isotype of purified antibodies, isotype ELISAs can be performed. For example, wells of microtiter plates can be coated with 10 μg/ml of anti-human Ig overnight at 4° C. After blocking with 5% BSA, the plates are reacted with 10 μg/ml of monoclonal antibodies or purified isotype controls, at ambient temperature for two hours. The wells can then be reacted with either human IgG1 or human IgM-specific alkaline phosphatase-conjugated probes. Plates are developed and analyzed as described above.

In order to demonstrate binding of monoclonal antibodies to live cells expressing the CD30, flow cytometry can be used. In a typical (but non-limiting) example of a flow cytometry protocol, cell lines expressing CD30 (grown under standard growth conditions) are mixed with various concentrations of monoclonal antibodies in PBS containing 0.1% BSA and 20% mouse serum, and incubated at 37° C. for 1 hour. After washing, the cells are reacted with Fluorescein-labeled anti-human IgG antibody under the same conditions as the primary antibody staining. The samples can be analyzed by FACScan instrument using light and side scatter properties to gate on single cells. An alternative assay using fluorescence microscopy may be used (in addition to or instead of) the flow cytometry assay. Cells can be stained exactly as described above and examined by fluorescence microscopy. This method allows visualization of individual cells, but may have diminished sensitivity depending on the density of the antigen.

Anti-CD30 human IgGs can be further tested for reactivity with CD30 antigen by Western blotting. For example, cell extracts from cells expressing CD30 can be prepared and subjected to sodium dodecyl sulfate (SDS) polyacrylamide gel electrophoresis. After electrophoresis, the separated antigens will be transferred to nitrocellulose membranes, blocked with 20% mouse serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

### Phagocytic and Cell Killing Activities of Human Monoclonal Antibodies to CD30

In addition to binding specifically to CD30, human monoclonal anti-CD30 antibodies can be tested for their ability to mediate phagocytosis and killing of cells expressing CD30. The testing of monoclonal antibody activity in vitro will provide an initial screening prior to testing in vivo models. Briefly, polymorphonuclear cells (PMN), or other effector cells, from healthy donors can be purified by Ficoll Hypaque density centrifugation, followed by lysis of contaminating erythrocytes. Washed PMNs, can be suspended in RPMI supplemented with 10% heat-inactivated fetal calf serum and mixed with ⁵¹Cr labeled cells expressing CD30, at various ratios of effector cells to tumor cells (-effector cells:tumor cells). Purified human anti-CD30 IgGs can then be added at various concentrations. Irrelevant human IgG can be used as negative control. Assays can be carried out for 4-18 hours at 37° C. Samples can be assayed for cytolysis by measuring ⁵¹Cr release into the culture supernatant. Anti-CD30 monoclonal can also be tested in combinations with each other to determine whether cytolysis is enhanced with multiple monoclonal antibodies.

Human monoclonal antibodies which bind to CD30 also can be tested in an in vivo model (e.g., in mice) to determine their efficacy in mediating phagocytosis and killing of cells expressing CD30, e.g., tumor cells. These antibodies can be selected, for example, based on the following criteria, which are not intended to be exclusive:

1.) binding to live cells expressing CD30;

2.) high affinity of binding to CD30;

3.) binding to a unique epitope on CD30 (to eliminate the possibility that monoclonal antibodies with complimentary activities when used in combination would compete for binding to the same epitope);

4.) opsonization of cells expressing CD30;

5.) mediation of growth inhibition, phagocytosis and/or killing of cells expressing CD30 in the presence of human effector cells.

Preferred human monoclonal antibodies of the invention meet one or more, and preferably all, of these criteria. In a particular embodiment, the human monoclonal antibodies are used in combination, e.g., as a pharmaceutical composition comprising two or more anti-CD30 monoclonal antibodies or fragments thereof. For example, human anti-CD30 monoclonal antibodies having different, but complementary activities can be combined in a single therapy to achieve a desired therapeutic or diagnostic effect. An illustration of this would be a composition containing an anti-CD30 human monoclonal antibody that mediates highly effective killing of target cells in the presence of effector cells, combined with another human anti-CD30 monoclonal antibody that inhibits the growth of cells expressing CD30.

### Bispecific/Multispecific Molecules Which Bind to CD30

In yet another embodiment of a method of the invention, human monoclonal antibodies to CD30, or antigen-binding portions thereof, can be derivatized or linked to another functional molecule, e.g., another peptide or protein (e.g., an Fab′ fragment) to generate a bispecific or multispecific molecule which binds to multiple binding sites or target epitopes. For example, an antibody or antigen-binding portion of the invention can be functionally linked (e.g., by chemical coupling, genetic fusion, noncovalent association or otherwise) to one or more other binding molecules, such as another antibody, antibody fragment, peptide or binding mimetic. Bispecific molecules useful in the present invention include those described in US 2004/0006215. In a particular embodiment, the bispecific antibody is H22×Ki4, which is also described in US 2004/0006215.

An “effector cell specific antibody” as used herein refers to an antibody or functional antibody fragment that binds the Fc receptor of effector cells. Preferred antibodies for use in the subject invention bind the Fc receptor of effector cells at a site which is not bound by endogenous immunoglobulin.

As used herein, the term “effector cell” refers to an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Some effector cells express specific Fc receptors and carry out specific immune functions. In preferred embodiments, an effector cell is capable of inducing antibody-dependent cell-mediated cytotoxicity (ADCC), e.g., a neutrophil capable of inducing ADCC. For example, monocytes, macrophages, which express FcR are involved in specific killing of target cells and presenting antigens to other components of the immune system, or binding to cells that present antigens. In other embodiments, an effector cell can phagocytose a target antigen, target cell, or microorganism. The expression of a particular FcR on an effector cell can be regulated by humoral factors such as cytokines. For example, expression of FcγRI has been found to be up-regulated by interferon gamma (IFN-γ). This enhanced expression increases the cytotoxic activity of FcγRI-bearing cells against targets. An effector cell can phagocytose or lyse a target antigen or a target cell.

“Target cell” shall mean any undesirable cell in a subject (e.g., a human or animal) that can be targeted by a composition (e.g., a human monoclonal antibody, a bispecific or a multispecific molecule) of the invention. In preferred embodiments, the target cell is a cell expressing or overexpressing CD30, e.g., a CD30 positive lymphoma. Cells expressing CD30 typically include tumor cells, such as bladder, breast, colon, kidney, ovarian, prostate, renal cell, squamous cell, lung (non-small cell), and head and neck tumor cells. Other target cells include synovial fibroblast cells.

Chimeric mouse-human monoclonal antibodies (i.e., chimeric antibodies) can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted. (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

The chimeric antibody can be further humanized by replacing sequences of the Fv variable region which are not directly involved in antigen binding with equivalent sequences from human Fv variable regions. General reviews of humanized chimeric antibodies are provided by Morrison, S. L., 1985, Science 229:1202-1207 and by Oi et al., 1986, BioTechniques 4:214. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from 7E3, an anti-GPII_{b}IIₐ antibody producing hybridoma. The recombinant DNA encoding the chimeric antibody, or fragment thereof, can then be cloned into an appropriate expression vector. Suitable humanized antibodies can alternatively be produced by CDR substitution U.S. Pat. No. 5,225,539; Jones et al. 1986 Nature 321:552-525; Verhoeyan et al. 1988 Science 239:1534; and Beidler et al. 1988 J. Immunol. 141:4053-4060.

All of the CDRs of a particular human antibody may be replaced with at least a portion of a non-human CDR or only some of the CDRs may be replaced with non-human CDRs. It is only necessary to replace the number of CDRs required for binding of the humanized antibody to the Fc receptor.

An antibody can be humanized by any method, which is capable of replacing at least a portion of a CDR of a human antibody with a CDR derived from a non-human antibody. Winter describes a method which may be used to prepare the humanized antibodies of the present invention (UK Patent Application GB 2188638A, filed on Mar. 26, 1987), the contents of which is expressly incorporated by reference. The human CDRs may be replaced with non-human CDRs using oligonucleotide site-directed mutagenesis as described in International Application WO 94/10332 entitled, Humanized Antibodies to Fc Receptors for Immunoglobulin G on Human Mononuclear Phagocytes.

Also within the scope of the invention are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances. Antibodies in which amino acids have been added, deleted, or substituted are referred to herein as modified antibodies or altered antibodies.

The term modified antibody is also intended to include antibodies, such as monoclonal antibodies, chimeric antibodies, and humanized antibodies which have been modified by, e.g., deleting, adding, or substituting portions of the antibody. For example, an antibody can be modified by deleting the constant region and replacing it with a constant region meant to increase half-life, e.g., serum half-life, stability or affinity of the antibody. Any modification is within the scope of the invention so long as the bispecific and multispecific molecule has at least one antigen binding region specific for an FcγR and triggers at least one effector function.

Bispecific and multispecific molecules of the present invention can be made using chemical techniques (see e.g., D. M. Kranz et al. (1981) Proc. Natl. Acad. Sci. USA 78:5807), “polydoma” techniques (See U.S. Pat. No. 4,474,893, to Reading), or recombinant DNA techniques.

In particular, bispecific and multispecific molecules of the present invention can be prepared by conjugating the constituent binding specificities, e.g., the anti-FcR and anti-CD30 binding specificities, using methods known in the art and described in the examples provided herein. For example, each binding specificity of the bispecific and multispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described by Paulus (Behring Ins. Mitt. (1985) No. 78, 118-132); Brennan et al. (Science (1985) 229:81-83), and Glennie et al. (J. Immunol. (1987) 139: 2367-2375). Preferred conjugating agents are SATA and sulfo-SMCC, both available from Pierce Chemical Co. (Rockford, Ill.).

When the binding specificities are antibodies (e.g. two humanized antibodies), they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific and multispecific molecule is a MAb×MAb, MAb×Fab, Fab×F(ab′)₂ or ligand×Fab fusion protein. A bispecific and multispecific molecule of the invention, e.g., a bispecific molecule can be a single chain molecule, such as a single chain bispecific antibody, a single chain bispecific molecule comprising one single chain antibody and a binding determinant, or a single chain bispecific molecule comprising two binding determinants. Bispecific and multispecific molecules can also be single chain molecules or may comprise at least two single chain molecules. Methods for preparing bi- and multispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Binding of the bispecific and multispecific molecules to their specific targets can be confirmed by enzyme-linked immunosorbent assay (ELISA), a radioimmunoassay (RIA), FACS analysis, a bioassay (e.g. growth inhibition), or a Western Blot Assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

### Immunoconjugates

In another aspect, antibodies used in the present invention can be conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other preferred examples of therapeutic cytotoxins that can be conjugated to an antibody of the invention include duocarmycins, calicheamicins, maytansines and auristatins, and derivatives thereof. An example of a calicheamicin antibody conjugate is commercially available (Mylotarg™; Wyeth-Ayerst).

Cytotoxins can be conjugated to antibodies used in the invention via linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Antibodies used in the present invention also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine¹³¹, indium¹¹¹, yttrium⁹⁰ and lutetium¹⁷⁷. Method for preparing radioimmunoconjugates are established in the art. Examples of radioimmunoconjugates are commercially available, including Zevalin™ (IDEC Pharmaceuticals) and Bexxar™ (Corixa Pharmaceuticals), and similar methods can be used to prepare radioimmunoconjugates using the antibodies of the invention.

The antibody conjugates used in a method of the invention can modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### Steroids

The present invention requires administration of a glucocorticosteroid in combination with an anti-CD30 antibody in order to treat a patient having a CD30 positive lymphoma. Any glucocorticoid, steroid analog, or salt thereof, which are known to chiefly influence carbohydrate, fat and protein metabolism, inhibit corticotropin secretion, and possess pronounced anti-inflammatory activity and immunosuppressive properties, within the class commonly known as glucocorticosteroids (e.g. cortisol, dexamethasone, prednisone, prednisolone, hydrocortisone, etc.) can be used in a method of the invention. The use of “glucocorticosteroid” herein is intended to include all compounds within the class.

Glucocorticoid steroids are well-known in the art, and include, but are not limited to, e.g., betamethasone, budesonide, cortisone, deflazacort, dexamethasone, hydrocortisone, hydrocortisone cypionate, methylprednisolone, prednisolone, prednisone, and triamcinolone. A method according to the present invention also includes use of a salt prepared from a glucocorticosteroid. Thus, reference to a particular glucocorticosteroid also includes the salt form.

### Pharmaceutical Compositions

Methods of the present invention employ (i) a composition, e.g., a pharmaceutical composition, containing one or a combination of anti-CD 30 monoclonal antibodies, or antigen-binding portion(s) thereof, of the present invention, formulated together with a pharmaceutically acceptable carrier and (ii) a composition, e.g., a pharmaceutical composition, containing a glucocorticosteroid formulated together with a pharmaceutically acceptable carrier. Antibody compositions may include one or a combination of (e.g., two or more different) antibodies, or immunoconjugates or bispecific molecules, as described herein. For example, an antibody pharmaceutical composition can comprise a combination of antibodies (or immunoconjugates or bispecifics) that bind to different epitopes on CD30 or that have complementary activities. An antibody and glucocorticosteroid used in a method of the present invention can also be formulated together if desirable.

A pharmaceutical compositions used in the invention also can be further combined with additional agents. For example, if desirable, the combination therapy of the invention, i.e., antibody and glucocorticosteroid can include one or more additional anti-tumor or cytostatic or cytotoxic agents. Examples of additional therapeutic agents that can be used in combination therapy are described in greater detail below in the section on uses of the antibodies of the invention.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, may be coated in a material to protect the compound from the action of acids and other natural conditions that may inactivate the compound.

The pharmaceutical compounds and compositions of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (13HA), butylated hydroxytoluene (BHT), lecithin, propyl gallate, alpha-tocopherol, and the like; and (3) metal chelating agents, such as citric acid, ethylenediamine tetraacetic acid (EDTA), deferoxamine (DEF), diethylenetriaminepentaacetic acid (DTPA), sorbitol, tartaric acid, phosphoric acid, histidine, and the like. Additional anti-oxidants that can be used are found in Akers, J. Parenteral Science and Technology 36: 222-228, 1982.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Pharmaceutical compositions used in a method of the invention may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. For oral administrations, the active compound is suitably protected and combined with an inert diluent or an assimilable edible carrier.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al. (1984) J. Neuroimmunol. 7:27).

In certain embodiments, the human monoclonal antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273. In one embodiment of the invention, the therapeutic compounds of the invention are formulated in liposomes; in a more preferred embodiment, the liposomes include a targeting moiety. In a most preferred embodiment, the therapeutic compounds in the liposomes are delivered by bolus injection to a site proximal to the desired area, e.g., the site of inflammation or infection, or the site of a tumor. The composition must be fluid to the extent that easy syringability exists. It must be stable under the conditions of manufacture and storage and must be preserved against the contaminating action of microorganisms such as bacteria and fungi.

The amount of active ingredient that can be combined with a carrier material to produce a single dosage form will vary depending upon the subject being treated, the particular mode of administration and the amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time, or the dose may be proportionally reduced or increased as indicated by the exigencies of the therapeutic situation. It is especially advantageous to formulate parenteral compositions in dosage unit form, which can be prepared according to well-known methods in the pharmaceutical arts, for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

A “therapeutically effective dosage” of an anti-CD30 antibody and a glucocorticosteroid used in the invention preferably results in a decrease in severity of disease symptoms, an increase in frequency and duration of disease symptom-free periods, and/or a prevention of impairment or disability due to the disease affliction. For example, for the treatment of CD30 positive tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors, e.g., as described in the Example, infra. Alternatively, this property of a composition can be evaluated by examining the ability of the compound to inhibit tumor growth in vitro by assays known to the skilled practitioner. Such assays are described, e.g., US Patent Application Publication No. 2004/0006215. A therapeutically effective amount of a therapeutic compound can decrease tumor size, or otherwise ameliorate symptoms in a subject. One of ordinary skill in the art can determine such amount based on factors such as the subject's size, the severity of the subject's symptoms, and the particular composition or route of administration selected.

For administration of the antibody, the dosage can range from about 0.0001 mg/kg to about 100 mg/kg of the host body weight, and more typically from about 0.1 mg/kg to about 50 mg/kg. For example, dosages can be 0.1 mg/kg body weight, 0.5 mg/kg body weight, 1 mg/kg body weight, 3 mg/kg body weight, 5 mg/kg body weight, 10 mg/kg body weight, 12.5 mg/kg body weight, 15 mg/kg body weight, 20 mg/kg body weight, 25 mg/kg body weight, 50 mg/kg body weight, or within the range of about 0.1 mg/kg to about 50 mg/kg.

It shall be appreciated by those of ordinary skill in the art that due to the variation in potency of glucocorticosteroids, the dosage of any particular glucocorticosteroid can be determined by its equivalent dosage relative to hydrocortisone. Thus, as used herein, “hydrocortisone equivalent” represents the amount of glucocorticosteroid (mg) to achieve the same potency as hydrocortisone. Table 1 provides guidance for determining hydrocortisone equivalents for several glucocorticosteroids (see also Goodman and Gilman, Pharmacological Basis of Therapeutics, 8^{th} ed., p. 1447 and Dubois, 2005, Curr. Resp. Med. Rev. 1: 103-108), which are hereby incorporated by reference in their entireties. It shall be appreciated by those of ordinary skill in the art that Table 1 is presented for purposes of guidance and that actual potency vary slightly.

TABLE 1

| | Relative potency in |
|---|---|
| Glucocorticoid | mg to hydrocortisone |
| Hydrocortisone | 1 |
| Betamethasone | 33.33 |
| Cortisol | 1.25 |
| Cortisone | 0.8 |
| Dexamethasone | 26.67 |
| Fludrocortisone | 15 |
| Methylprednisolone | 5 |
| Prednisolone | 4 |
| Prednisone | 4 |
| Triamcinolone | 5 |

Thus, for administration of the glucocorticosteroid, the dosage can range from about 0.01 mg to about 10,000 mg hydrocortisone equivalent, preferably from about 1 mg to about 5,000 mg hydrocortisone equivalent, more preferably from about 10 mg to about 2500 mg hydrocortisone equivalent, still more preferably from about 40 mg to about 2000 mg hydrocortisone equivalent, and most preferably from about 80 mg to about 1600 mg hydrocortisone equivalent. In particular embodiments, described in the Example infra, about 160 mg to about 1400 mg hydrocortisone equivalent is administered to patients according to various treatment regimens.

In a particular embodiment of a method of the invention, prednisolone or prednisone can be administered in a dosage range from about 1 mg to about 200 mg, more preferably from about 10 mg to about 160 mg, and most preferably from about 20 to about 120 mg.

In another particular embodiment, dexamethasone can be administered in a dosage range from about 1 mg to about 100 mg, more preferably from about 5 mg to about 80 mg, and most preferably from about 20 mg to about 60 mg.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a compositions of the invention will be that amount of the compound which is the lowest dose effective to produce a therapeutic effect. Such an effective dose will generally depend upon the factors described above. If desired, the effective daily dose of a therapeutic compositions may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

The administration of the antibody vis-à-vis glucocorticosteroid can be carried out according to a variety of schedules. For example, the antibody and glucocorticosteroid compositions can be administered concurrently, i.e., a glucocorticosteroid is administered within the same week as (or within 7 days of) antibody treatment or glucocorticosteroid is administered between two or more administrations of the anti-CD30 antibody.

Alternatively, the glucocorticosteroid can be administered prior to the first administration of the antibody. In this regard, the first administration of the glucocorticosteroid can begin one, two, three, four, five, six, seven, eight, or more weeks prior to the first administration of the antibody. Glucocorticosteroid administration can be stopped prior to the first administration of the antibody or it can be continued during the course of administration for the antibody (i.e., administered concurrently). In the case of the latter, the glucocorticosteroid administration can be stopped when antibody treatment is stopped or continued until some time after antibody treatment is completed, e.g. from 1 week to 6 or more months following completion of antibody therapy depending on the needs of the patient being treated.

In another embodiment, a first glucocorticosteroid administration can be given more than 1 week after the end of antibody treatment and continued for 1 week to 6 or more months depending on the needs of the patient being treated.

In any of the treatment schedules described above, administration of the antibody and glucocorticosteroid occurs within 3 months of each other, i.e., at least one administration of either the antibody or the glucocorticosteroid must occur within 3 months of the other. Preferably, administration of the antibody and glucocorticosteroid occurs within 2 months of each other, more preferably administration of the antibody and glucocorticosteroid occurs within 1 month of each other, still more preferably administration of the antibody and glucocorticosteroid occurs within 3 weeks of each other, still more preferably administration of the antibody and glucocorticosteroid occurs within 2 weeks of each other, and most preferably administration of antibody and glucocorticosteroid is concurrent. It shall be appreciated by those of ordinary skill in the art that the schedules described herein can be tailored to meet the needs of the patient being treated.

Regardless of the schedule selected for administering the combination, the antibody can be administered once per week, once every two weeks, once every three weeks, once every four weeks or once a month, once every 3 months or once every 3 to 6 months. Treatment can also continue until the patient exhibits a clinical response, or treatment can be reinitiated if the patient relapses. Preferred dosage regimens for an anti-CD30 antibody of the invention include 1 mg/kg body weight, 3 mg/kg body weight, 5 mg/kg body weight, 10 mg/kg body weight, 15 mg/kg body weight and 25 mg/kg body weight via intravenous administration, with the antibody being given using, e.g., one of the following dosing schedules: (i) every four weeks for six dosages, then every three months; (ii) every three weeks; (iii) a higher dose, e.g., 10 to 25 mg/kg body weight once followed by a lower dose, e.g., 1 to 5 mg/kg body weight every three weeks. The glucorticosteroids can be administered according to conventional steroid regimens well-known in the art for treating cancers, e.g., once per day for 1 to 5 days every 3 to 4 weeks or every other day for 1 week to 6 months. In some cases, it may be desirable to administer a large dose of the glucocorticosteroid in a single day, e.g., about 1,400 mg hydrocortisone equivalent or more (e.g., 50 mg dexamethasone).

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

Antibody and glucocorticosteroid can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic, therapeutic or maintenance. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a maintenance regimen, which might entail a similar dosage schedule as used in treatment but with lower dosages, in order to prevent regrowth of the tumor in order to maintain the initial response.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Pharmaceutical compositions used in the present invention can be administered via one or more routes of administration using one or more of a variety of methods known in the art. As will be appreciated by the skilled artisan, the route and/or mode of administration will vary depending upon the desired results. Preferred routes of administration for antibodies of the invention include intravenous, intramuscular, intradermal, intraperitoneal, subcutaneous, spinal or other parenteral routes of administration, for example by injection or infusion. The phrase “parenteral administration” and “administered parenterally”, as used herein, means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. A glucocorticosteroid used in the present method can be administered in any one or more of the aforementioned routes.

Alternatively, an antibody or glucocorticosteroid used in a method of the invention can be administered via a non-parenteral route, such as a topical, epidermal or mucosal route of administration, for example, intranasally, orally, vaginally, rectally, sublingually or topically. Preferably a glucocorticosteroid is administered orally.

Therapeutic compositions can be administered with medical devices known in the art. For example, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

For example, the human antibodies, antibody compositions, glucocorticosteroids and methods of the present invention can be used to treat a subject with a tumorigenic disorder, e.g., a disorder characterized by the presence of tumor cells expressing CD30 including B cell and T cell lymphomas, for example, Hodgkin's disease, anaplastic large cell lymphoma (ALCL), adult T-cell lymphoma. (ATL), angioimmunoblastic lymphadenopathy (AILD)-like T cell lymphoma, HIV associated body cavity based lymphomas, Embryonal Carcinomas, undifferentiated carcinomas of the rhino-pharynx (e.g., Schmincke's tumor), Castleman's disease, Kaposi's Sarcoma and other T-cell or B-cell lymphomas. The human antibodies, antibody compositions and the methods of the present invention can also be used to treat a subject with other disorders, e.g., autoimmune diseases, including, for example, Rheumatoid arthritis, Systemic Lupus Erythematosus, Systemic Sclerosis, Atopic Dermatitis, Graves' disease, Hashimoto's thyroiditis, Wegner's granulomatosis, Omen's syndrome, chronic renal failure, acute infectious mononucleosis, HIV and herpes virus associated diseases.

In a particular embodiment, a method of the present invention is used in vivo to treat, prevent or diagnose a variety of CD30-related diseases. Examples of CD30-related diseases include, among others, cancer, Hodgkin's disease, non-Hodgkin's lymphoma, anaplastic large cell lymphoma (ALCL), adult T-cell lymphoma. (ATL), angioimmunoblastic lymphadenopathy (AILD)-like T cell lymphoma, HIV associated body cavity based lymphomas, Embryonal Carcinomas, undifferentiated carcinomas of the rhino-pharynx (e.g., Schmincke's tumor), Castleman's disease, Kaposi's Sarcoma and other T-cell or B-cell lymphomas. Other CD30 mediated diseases include among others, autoimmune diseases, Rheumatoid arthritis, Systemic Lupus Erythematosus, Systemic Sclerosis, Atopic Dermatitis, Graves' disease, Hashimoto's thyroiditis, Wegner's granulomatosis, Omen's syndrome, chronic renal failure, acute infectious mononucleosis, HIV and herpes virus associated diseases.

In a particular embodiment, a method of the present invention is used to treat or to prevent Hodgkin's disease (HD), as the antibodies limit the role that CD30 plays in the progression of HD and other tumorigenic diseases. Hodgkin's disease is a type of lymphoma. Lymphomas are cancers that develop in the lymph system, part of the body's immune system. Because there is lymph tissue in many parts of the body, HD can start in almost any part of the body. The cancer can spread to almost any organ or tissue in the body, including the liver, bone marrow (the spongy tissue inside the large bones of the body that makes blood cells), and the spleen. Elevated expression of CD30 in Hodgkin's and Reed-Sternberg cells has been reported to correlate with the differential diagnosis of HD. Accordingly, CD30 inhibiting antibodies in combination with a glucocorticosteroid can be used to prevent or block the effects of CD30 which lead to HD and, thus, can be used to prevent or treat this disease.

Human antibodies (e.g., human monoclonal antibodies, multispecific and bispecific molecules) in combination with glucorticosteroids also can be used to block or inhibit other effects of CD30. For example, it is known that CD30 is also regularly expressed by a variety of non-Hodgkin's lymphoma subtypes. Accordingly, yet another use for the method of the invention includes the prevention or treatment of diseases involving non-Hodgkin's lymphomas, e.g., any CD30-positive B or T cell lymphoma. These diseases include Burkitt's lymphoma, anaplastic large-cell lymphomas (ALCL), cutaneous T-cell lymphomas, nodular small cleaved-cell lymphomas, lymphocytic lymphomas, peripheral T-cell lymphomas, Lennert's lymphomas, immunoblastic lymphomas, T-cell leukemia/lymphomas (ATLL), adult T-cell leukemia (T-ALL), and entroblastic/centrocytic (cb/cc) follicular lymphomas cancers.

In another particular embodiment, a method of the present invention can be used to block or inhibit yet other effects of CD30. For example, it is also known that soluble CD30 is regularly shed from the surface of cells expressing CD30. Elevated sCD30 levels have been reported in the serum of patients with a variety of tumorigenic and autoimmune disorders. Accordingly, yet another use for the anti-CD30-antibodies in combination with a glucocorticosteroid includes the prevention or treatment of diseases involving blocking or inhibiting of shedding of sCD30. Such diseases include, but are not limited to, Rheumatoid arthritis, Systemic Lupus Erythematosus, Systemic Sclerosis, Atopic Dermatitis, Graves' disease, Hashimoto's thyroiditis, Wegner's granulomatosis, and Omen's syndrome.

As previously described, human anti-CD30 antibodies and glucorticosteroids used in the invention can be co-administered with one or more other therapeutic agents, e.g., a cytotoxic agent, a radiotoxic agent, or an immunosuppressive agent. The antibody can be linked to the agent (as an immunocomplex) or it can be administered separate from the agent. In the latter case (separate administration), the antibody can be administered before, after or concurrently with the agent or it can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil, and cyclophosphamide hydroxyurea which, by themselves, are only effective at levels which are toxic or subtoxic to a patient. Cisplatin is intravenously administered as a 100 mg/m² dose once every four weeks and adriamycin is intravenously administered as a 60-75 mg/m² dose once every 21 days. Co-administration with other chemotherapeutic agents provides two anti-cancer agents which operate via different mechanisms in order to yield a cytotoxic effect to human tumor cells. Such co-administration can solve problems due to development of resistance to drugs or a change in the antigenicity of the tumor cells which would render them unreactive with the antibody.

Target-specific effector cells, e.g., effector cells linked to compositions (e.g., human antibodies, multispecific and bispecific molecules) of the invention can also be used as therapeutic agents. Effector cells for targeting can be human leukocytes such as macrophages, neutrophils or monocytes. Other cells include eosinophils, natural killer cells and other IgG- or IgA-receptor bearing cells. If desired, effector cells can be obtained from the subject to be treated. The target-specific effector cells, can be administered as a suspension of cells in a physiologically acceptable solution. The number of cells administered can be in the order of 10⁸-10⁹ but will vary depending on the therapeutic purpose. In general, the amount will be sufficient to obtain localization at the target cell, e.g., a tumor cell expressing CD30, and to effect cell killing by, e.g., phagocytosis. Routes of administration can also vary.

Therapy with target-specific effector cells can be performed in conjunction with other techniques for removal of targeted cells. For example, anti-tumor therapy using the compositions (e.g., human antibodies, multispecific and bispecific molecules) of the invention and/or effector cells armed with these compositions can be used in conjunction with chemotherapy. Additionally, combination immunotherapy may be used to direct two distinct cytotoxic effector populations toward tumor cell rejection. For example, anti-CD30 antibodies linked to anti-Fc-gamma RI or anti-CD3 may be used in conjunction with IgG- or IgA-receptor specific binding agents.

Bispecific and multispecific molecules derived from anti-CD30 antibodies can also be used to modulate FcγR or FcαR levels on effector cells, such as by capping and elimination of receptors on the cell surface. Mixtures of anti-Fc receptors can also be used for this purpose.

The anti-CD30 antibody pharmaceutical compositions used in the invention which have complement binding sites, such as portions from IgG1, -2, or -3 or IgM which bind complement, can also be used in the presence of complement. In one embodiment, ex vivo treatment of a population of cells comprising target cells with a binding agent of the invention and appropriate effector cells can be supplemented by the addition of complement or serum containing complement. Phagocytosis of target cells coated with a binding agent of the invention can be improved by binding of complement proteins. In another embodiment target cells coated with the compositions (e.g., human antibodies, multispecific and bispecific molecules) of the invention can also be lysed by complement. In yet another embodiment, the compositions of the invention do not activate complement.

The antibody compositions (e.g., human antibodies, multispecific and bispecific molecules and immunoconjugates) of the invention can also be administered together with complement. Accordingly, within the scope of the invention are compositions comprising human antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules of the invention and the complement or serum can be administered separately.

Accordingly, patients treated with antibody and glucocorticosteroid compositions of the invention can be additionally administered (prior to, concurrently with, or following administration of a human antibody of the invention) with another therapeutic agent, such as a cytotoxic or radiotoxic agent, which enhances or augments the therapeutic effect of the human antibodies.

In other embodiments, the subject can be additionally treated with an agent that modulates, e.g., enhances or inhibits, the expression or activity of Fcγ or Fcα receptors by, for example, treating the subject with a cytokine. Preferred cytokines for administration during treatment with the multispecific molecule include of granulocyte colony-stimulating factor (G-CSF), granulocyte-macrophage colony-stimulating factor (GM-CSF), interferon-γ (IFN-γ), and tumor necrosis factor (TNF).

In another embodiment, the subject can be additionally treated with a lymphokine preparation. Cancer cells which do not highly express CD30 can be induced to do so using lymphokine preparations. Lymphokine preparations can cause a more homogeneous expression of CD30s among cells of a tumor which can lead to a more effective therapy. Lymphokine preparations suitable for administration include interferon-gamma, tumor necrosis factor, and combinations thereof. These can be administered intravenously. Suitable dosages of lymphokine are 10,000 to 1,000,000 units/patient.

The antibody and glucocorticosteroid compositions used in the invention can also be used to target cells expressing FcγR or CD30, for example for labeling such cells. For such use, the binding agent can be linked to a molecule that can be detected. Thus, the invention provides methods for localizing ex vivo or in vitro cells expressing Fc receptors, such as FcγR, or CD30. The detectable label can be, e.g., a radioisotope, a fluorescent compound, an enzyme, or an enzyme co-factor.

In other embodiments, the invention provides methods for treating a CD30 mediated disorder in a subject, e.g., Hodgkin's disease, adult T-cell lymphoma, infectious mononucleosis, and Systemic Lupus Erythematosus, by administering to the subject an anti-CD30 antibody and a glucocorticosteroid as described above. Such antibodies and derivatives thereof are used to inhibit CD30 induced activities associated with certain disorders, e.g., proliferation and differentiation. Other CD30 induced activities which can be inhibited by the antibodies of the present invention include increased production of sCD30, increased expression of IL-4 and increased production of the Th2 phenotype. By contacting the antibody with CD30 (e.g., by administering the antibody to a subject), the ability of CD30 to induce such activities is inhibited and, thus, the associated disorder is treated. Preferred antibodies bind to epitopes which are specific to CD30 and, thus, advantageously inhibit CD30 induced activities, but do not interfere with the activity of structurally related surface antigens, such as NGFR, CD27 and CD40.

Accordingly, in another embodiment, the present invention provides a method for treating or preventing a tumorigenic disorder mediated by human CD30, e.g., Hodgkin's disease, non-Hodgkin's lymphoma, anaplastic large cell lymphoma (ALCL), adult T-cell lymphoma. (ATL), angioimmunoblastic lymphadenopathy (AILD)-like T cell lymphoma, HIV associated body cavity based lymphomas, Embryonal Carcinomas, undifferentiated carcinomas of the rhino-pharynx (e.g., Schmincke's tumor), Castleman's disease, Kaposi's Sarcoma and other T-cell or B-cell lymphomas. The method involves administering to a subject an antibody composition of the present invention in an amount effective to treat or prevent the disorder. The antibody and glucocorticosteroid compositions can be administered along with another therapeutic agent, such as a cytotoxic or a radiotoxic agent which acts in conjunction with or synergistically with the antibody composition to treat or prevent the CD30 mediated disease. In a particularly preferred embodiment, the present invention provides a method for treating Hodgkin's disease. In yet another particularly preferred embodiment, the present invention provides a method for treating ALCL.

In another embodiment, the present invention provides a method for treating or preventing an autoimmune disorder mediated by human CD30, e.g., rheumatoid arthritis, Systemic Lupus Erythematosus, Systemic Sclerosis, Atopic Dermatitis, Graves' disease, Hashimoto's thyroiditis, Wegner's granulomatosis, Omen's syndrome, chronic renal failure, acute infectious mononucleosis, HIV and herpes virus associated diseases. The method involves administering to a subject an antibody and glucocorticosteroid compositions of the present invention in amounts effective to treat or prevent the disorder. The compositions can be administered alone or along with another therapeutic agent, such as an immunosuppressant which acts in conjunction with or synergistically with the antibody composition to treat or prevent the CD30 mediated disease.

In yet another embodiment, immunoconjugates of the invention can be used in combination with a glucocorticosteroid to target compounds (e.g., therapeutic agents, labels, cytotoxins, radiotoxoins immunosuppressants, etc.) to cells which have CD30 bound to their surface (e.g., membrane bound or bound to CD30 receptor) by linking such compounds to the antibody. Thus, the invention also provides methods for localizing ex vivo or in vitro cells expressing CD30 and CD30 receptor, such as Hodgkin's cells or Reed-Sternberg cells (e.g., with a detectable label, such as a radioisotope, a fluorescent compound, an enzyme, or an enzyme co-factor). Alternatively, the immunoconjugates can be used to kill cells which have CD30 bound to their surface (e.g., membrane bound or bound to CD30 receptor) by targeting cytotoxins or radiotoxins to CD30.

The present invention is further illustrated by the following examples which should not be construed as further limiting.

### EXAMPLE

The following Example demonstrates the efficacy of a combination therapy of anti-CD30 antibody and glucocorticosteroid in treating CD30 positive lymphomas, including Hodgkin's Disease (HD) and T cell lymphoma.

### Patient 1

The patient, diagnosed with HD, received 5 mg/kg anti-CD30 antibody 5F11 by i.v. infusion once per day per week for 3 weeks. The week after completing antibody therapy (i.e., fourth week from start of therapy), the patient received 40 mg oral dexamethasone once per day for 2 days; one month later the patient received another 40 mg oral dexamethasone once per day for 4 days; two weeks later the patient received a third regimen of 40 mg oral dexamethasone once per day for 4 days. This patient achieved a partial response.

### Patient 2

Patient 2, diagnosed with HD, received 15 mg/kg anti-CD30 antibody 5F11 by i.v. infusion once per day per week for 3 weeks. At the start of week 3 of antibody therapy, the patient received one dose of 50 mg oral dexamethasone. After completion of antibody therapy (i.e., at the beginning of week four following the start of therapy), the patient received one dose of 100 mg oral prednisolone. This patient achieved a partial response, which improved to complete a response.

### Patient 3

Patient 3, diagnosed with T cell lymphoma, received 40 mg oral dexamethasone once per day for 4 days. On day 5, dexamethasone treatment ceased and 50 mg oral prednisolone was administered once per day for 20 days. At the beginning of the second week of steroid therapy, antibody therapy was initiated by administering 15 mg/kg anti-CD30 antibody 5F11 by i.v. infusion once per day per week for 3 weeks. This patient achieved a complete response.

### Patient 4

Patient 4, diagnosed with HD, received 1 mg/kg anti-CD30 antibody 5 μl by i.v. infusion once per day per week for 3 weeks. On the second day of week 1 after beginning therapy, the patient received 40 mg oral dexamethasone once per day for 4 days, followed by no steroid therapy for 10 days. Thus, a dexamethasone cycle was completed over a 14 day period. For this patient, 8 cycles of dexamethasone therapy were completed over a period of 4 months. This patient achieved a partial response.

### EQUIVALENTS

Those skilled in the art will recognize, or be able to ascertain using no more than routine experimentation, many equivalents of the specific embodiments of the invention described herein. Such equivalents are intended to be encompassed by the following claims.

### INCORPORATION BY REFERENCE

All patents, pending patent applications and other publications cited herein are hereby incorporated by reference in their entirety.

We claim:

1. A method of treating a CD30 positive lymphoma by administering to a patient in need of such treatment therapeutically effective amounts of (i) a monoclonal antibody that binds CD30 and (ii) a glucocorticosteroid.

2. A method of treating a CD30 positive lymphoma by administering to a patient in need of such treatment therapeutically effective amounts of (i) a monoclonal antibody that binds CD30 and (ii) a glucocorticosteroid, wherein the glucocorticosteroid improves the efficacy of the antibody.

3. The method of claim 2, wherein the improvement in the efficacy of the antibody is due to a synergistic or additive effect between the antibody and the glucocorticosteroid.

4. The method of any of the preceding claims, wherein the glucocorticosteroid is selected from betamethasone, budesonide, cortisol, cortisone, deflazacort, dexamethasone, hydrocortisone, hydrocortisone cypionate, methylprednisolone, prednisolone, prednisone, triamcinolone, and pharmaceutically acceptable salts thereof.

5. The method of any of the preceding claims, wherein the antibody is selected from 17G1, 2H9, 5F11, M44, HeFi-1, C10, AC10, Ber-H2, HRS-1, HRS-3, HRS-4, Ki-1, Ki-2, Ki-3, Ki-4, Ki-5, Ki-6, Ki-7, IRac, M67, T6, T13, T14, T24, T25, and an anti-CD30 antibody that competes for binding with 17G1, 2H9, 5F11, M44, HeFi-1, C10, AC10, Ber-H2, HRS-1, HRS-3, HRS-4, Ki-1, Ki-2, Ki-3, Ki-4, Ki-5, Ki-6, Ki-7, IRac, M67, T6, T13, T14, T24, or T25.

6. The method of any of the preceding claims, wherein the antibody is 5F11 and the glucocorticosteroid selected is selected from dexamethasone, prednisone, prednisolone, and pharmaceutically acceptable salts thereof.

7. The method of any of the preceding claims, wherein the patient receives a first administration of the glucocorticosteroid prior to a first administration of the antibody.

8. The method of any of the preceding claims, wherein the patient receives one or more administration of the glucocorticosteroid subsequent to the first administration of the antibody.

9. The method of any of the preceding claims, wherein the patient receives a first administration of the antibody prior to a first administration of the glucocorticosteroid.

10. The method of claim 9, wherein the patient receives one or more administrations of the antibody subsequent to the first administration of the glucocorticosteroid.

11. The method of any one of claims 1-6, wherein the antibody and glucocorticosteroid are administered concurrently.

12. The method of any of the preceding claims, wherein the dosage of the antibody is from about 0.0001 to about 100 mg/kg.

13. The method of any of the preceding claims, wherein the dosage of the antibody is from about 0.1 mg/kg to about 50 mg/kg.

14. The method of any of the preceding claims, wherein the dosage of the antibody is from about 1 mg/kg to about 25 mg/kg.

15. The method of any of the preceding claims, wherein the dosage of the glucorticosteroids is from about 0.01 mg to about 10,000 mg hydrocortisone equivalent per dose.

16. The method of any of the preceding claims, wherein the dosage of the glucocorticosteroid is from about 1 mg to about 5,000 mg hydrocortisone equivalent per dose.

17. The method of any of the preceding claims, wherein the dosage of the glucocorticosteroid is from about 80 mg to about 1,600 mg hydrocortisone equivalent per dose.

18. The method of any of the preceding claims, wherein the antibody comprises a human IgGlheavy chain or a human IgG4 heavy chain.

19. The method of any of the preceding claims, wherein the antibody comprises a human IgG heavy chain and a human kappa light chain.

20. The method of any of the preceding claims, wherein the monoclonal antibody comprises a human heavy chain variable region comprising FR1, CDR1, FR2, CDR2, FR3, CDR3 and FR4 sequences and a human light chain variable region comprising FR1, CDR1, FR2, CDR2, FR3, CDR3 and FR4 sequences, wherein:
(a) the human heavy chain variable region CDR3 sequence is selected from the group consisting of SEQ ID NOs: 18, 30 and 42, and conservative sequence modifications thereof;
(b) the human light chain variable region CDR3 sequence is selected from the group consisting of SEQ ID NOs: 24, 36 and 48, and conservative sequence modifications thereof; and
(c) the antibody binds to human CD30 with an affinity constant of at least 10⁷ M⁻¹.

21. The method of claim 20, wherein the human heavy chain variable region CDR2 sequence is selected from the group consisting of SEQ ID NOs: 17, 29 and 41, and conservative sequence modifications thereof; and the human light chain variable region CDR2 sequence is selected from the group consisting of SEQ ID NOs: 23, 35 and 47, and conservative sequence modifications thereof.

22. The method of claim 21, wherein the human heavy chain variable region CDR1 sequence is selected from the group consisting of SEQ ID NOs: 16, 28 and 40, and conservative sequence modifications thereof; and the human light chain variable region CDR1 sequence is selected from the group consisting of SEQ ID NOs: 22, 34 and 46, and conservative sequence modifications thereof.

23. The method of claim 20, wherein the antibody binds to human CD30 with an affinity constant of at least 10⁸ M⁻¹.

24. The method of claim 20, wherein the antibody binds to human CD30 with an affinity constant of at least 10⁹ M⁻¹.

25. The method of claim 20, wherein the human heavy chain variable region FR1, FR2, FR3 and FR4 sequences are derived from the human heavy chain VH₄₋₃₄ or VH₃₋₁₁ germline sequence.

26. The method of claim 20, wherein the human light chain variable region FR1, FR2, FR3 and FR4 sequences are derived from the human light chain L15, A27 or L6 germline sequence.

27. The method of any of the preceding claims wherein the antibody comprises a human heavy chain variable region and a human light chain variable region, wherein:
(a) the human heavy chain variable region comprises an amino acid sequence selected from the group consisting of SEQ ID NOs: 2, 6, 10, and sequences that are at least 80% homologous to SEQ ID NOs: 2, 6 and 10;
(b) the human light chain variable region comprises an amino acid sequence selected from the group consisting of SEQ ID NOs: 4, 8, 12, and sequences that are at least 80% homologous to SEQ ID NOs: 4, 8 and 12; and
(c) the human antibody binds to human CD30 with an affinity constant of at least 10⁷ M⁻¹.

28. The method of claim 27, wherein the antibody binds to human CD30 with an affinity constant of at least 10⁸ M⁻¹.

29. The method of claim 27, wherein the antibody binds to human CD30 with an affinity constant of at least 10⁹ M⁻¹.

30. The method of any of the preceding claims, wherein the antibody comprises a human heavy chain variable region derived from the human heavy chain VH₄₋₃₄ germline sequence and a human light chain variable region derived from the human light chain L15 germline sequence, wherein:
(a) the human heavy chain variable region comprises the amino acid sequence of SEQ ID NO: 10 or a sequence that is at least 80% homologous to SEQ ID NO: 10;
(b) the human light chain variable region comprises the amino acid sequence of SEQ ID NO: 12 or a sequences that is at least 80% homologous to SEQ ID NO: 12; and
(c) the human antibody binds to human CD30 with an affinity constant of at least 10⁷ M⁻¹.

31. The method of any one of claims 1-29 wherein the antibody comprises human heavy chain and human light chain variable regions comprising the amino acid sequences shown in SEQ ID NO:2 and SEQ ID NO:4, respectively.

32. The method of any one of claims 1-29 wherein the antibody comprises human heavy chain and human light chain variable regions comprising the amino acid sequences shown in SEQ ID NO: 6 and SEQ ID NO:8, respectively.

33. The method of any one of claims 1-29, wherein the antibody comprises human heavy chain and human light chain variable regions comprising the amino acid sequences shown in SEQ ID NO: 10 and SEQ ID NO:12, respectively.

34. The method of any of the preceding claims, where the antibody is produced by a hybridoma, wherein the hybridoma is prepared from a B cell obtained from a transgenic non-human animal having a genome comprising a human heavy chain transgene or transchromosome and a human light chain transgene or transchromosome, fused to an immortalized cell.

35. A method of treating a disease characterized by growth of tumor cells expressing CD30, comprising administering to a patient a monoclonal antibody that binds CD30 and a glucocorticosteroid.

36. A method of treating a disease characterized by growth of tumor cells expressing CD30, comprising administering to a patient a monoclonal antibody that binds CD30 and a glucocorticosteroid, wherein the glucocorticosteroid improves the efficacy of the antibody.

37. The method of claim 36, wherein the improvement in the efficacy of the antibody is due to a synergistic or additive effect between the antibody and the glucocorticosteroid.

38. The method of any one of claims 35-37, wherein the disease is selected from the group consisting of Hodgkin's disease, anaplastic large cell lymphoma (ALCL), adult T-cell lymphoma (ATL), angioimmunoblastic lymphadenopathy (AILD)-like T cell lymphoma, HIV associated body cavity based lymphomas, Embryonal Carcinomas, undifferentiated carcinomas of the rhino-pharynx (e.g., Schmincke's tumor), Castleman's disease, Kaposi's Sarcoma and other T-cell or B-cell lymphomas.

39. The method of claim any one of claims 35-37, wherein the disease is Hodgkin's disease.

40. The method of claim any one of claims 35-37, wherein the disease is non-Hodgkin's lymphoma.

41. The method of claim any one of claims 35-37, wherein the non-Hodgkin's lymphoma is anaplastic large cell lymphoma (ALCL).

42. A composition comprising a monoclonal antibody that binds CD30 and a synergistic amount of a glucocorticosteroid.

43. The composition of claim 42, wherein the glucocorticosteroid is selected from betamethasone, budesonide, cortisol, cortisone, deflazacort, dexamethasone, hydrocortisone, hydrocortisone cypionate, methylprednisone, prednisolone, prednisone, triamcinolone, and pharmaceutically acceptable salts thereof.

44. The composition of claim 42 or 43, wherein the antibody is selected from 17G1, 2H9, 5F11, M44, HeFi-1, C10, AC10, Ber-H2, HRS-1, HRS-3, HRS-4, Ki-1, Ki-2, Ki-3, Ki-4, Ki-5, Ki-6, Ki-7, IRac, M67, T6, T13, T14, T24, T25, and an anti-CD30 antibody that competes for binding with 17G1, 2H9, 5F11, M44, HeFi-1, C10, AC10, Ber-H2, HRS-1, HRS-3, HRS-4, Ki-1, Ki-2, Ki-3, Ki-4, Ki-5, Ki-6, Ki-7, IRac, M67, T6, T13, T14, T24 or T25.

45. The composition of any one of claims 42-44, wherein the antibody is 5F11 and the glucocorticosteroid selected is selected from dexamethasone, prednisone, prednisolone, and pharmaceutically acceptable salts thereof.

46. The composition of any one of claims 42-45, wherein the antibody comprises a human IgGlheavy chain or a human IgG4 heavy chain.

47. The composition of any one of claims 42-46, wherein the antibody comprises a human IgG heavy chain and a human kappa light chain.

48. The composition of any one of claims 42-47, wherein the monoclonal antibody comprises a human heavy chain variable region comprising FR1, CDR1, FR2, CDR2, FR3, CDR3 and FR4 sequences and a human light chain variable region comprising FR1, CDR1, FR2, CDR2, FR3, CDR3 and FR4 sequences, wherein:
(a) the human heavy chain variable region CDR3 sequence is selected from the group consisting of SEQ ID NOs: 18, 30 and 42, and conservative sequence modifications thereof;
(b) the human light chain variable region CDR3 sequence is selected from the group consisting of SEQ ID NOs: 24, 36 and 48, and conservative sequence modifications thereof; and
(c) the antibody binds to human CD30 with an affinity constant of at least 10⁷ M⁻¹.

49. The composition of claim 48, wherein the human heavy chain variable region CDR2 sequence is selected from the group consisting of SEQ ID NOs: 17, 29 and 41, and conservative sequence modifications thereof; and the human light chain variable region CDR2 sequence is selected from the group consisting of SEQ ID NOs: 23, 35 and 47, and conservative sequence modifications thereof.

50. The composition of claim 49, wherein the human heavy chain variable region CDR1 sequence is selected from the group consisting of SEQ ID NOs: 16, 28 and 40, and conservative sequence modifications thereof; and the human light chain variable region CDR1 sequence is selected from the group consisting of SEQ ID NOs: 22, 34 and 46, and conservative sequence modifications thereof.

51. The composition of any one of claims 42-50 wherein the antibody comprises a human heavy chain variable region and a human light chain variable region, wherein:
(a) the human heavy chain variable region comprises an amino acid sequence selected from the group consisting of SEQ ID NOs: 2, 6, 10, and sequences that are at least 80% homologous to SEQ ID NOs: 2, 6 and 10;
(b) the human light chain variable region comprises an amino acid sequence selected from the group consisting of SEQ ID NOs: 4, 8, 12, and sequences that are at least 80% homologous to SEQ ID NOs: 4, 8 and 12; and
(c) the human antibody binds to human CD30 with an affinity constant of at least 10⁷ M⁻¹.

52. The composition of any one of claims 42-51 wherein the antibody comprises human heavy chain and human light chain variable regions comprising the amino acid sequences shown in SEQ ID NO:2 and SEQ ID NO:4, respectively.

53. The composition of any one of claims 42-51 wherein the antibody comprises human heavy chain and human light chain variable regions comprising the amino acid sequences shown in SEQ ID NO: 6 and SEQ ID NO:8, respectively.

54. The composition of any one of claims 42-51, wherein the antibody comprises human heavy chain and human light chain variable regions comprising the amino acid sequences shown in SEQ ID NO: 10 and SEQ ID NO:12, respectively.

55. The composition of any one of claims 42-54, where the antibody is produced by a hybridoma, wherein the hybridoma is prepared from a B cell obtained from a transgenic non-human animal having a genome comprising a human heavy chain transgene or transchromosome and a human light chain transgene or transchromosome, fused to an immortalized cell.
